(19) United States
(12) Patent Application Publication
(10) Pub. No.: US 2012/0282167 A1
(43) Pub. Date: Nov. 8, 2012

(54) METHOD FOR PREDICTING THE SENSITIVITY OF A TUMOR TO AN EPIGENETIC TREATMENT

(75) Inventors: Céline Vallot, Paris (FR); Francois Radvanyi, Fontenay Aux Roses (FR); Nicolas Stransky, Cambridge, MA (US); Yves Allory, Paris (FR)

(73) Assignees: INSTITUT CURIE, PARIS CEDEX 05 (FR); CENTRE NATIONAL DE LA RECHERCHE SCIENTIFIQUE, PARIS CEDEX 16 (FR); ASSISTANCE PUBLIQUE-HOPITAUX DE PARIS, PARIS (FR); UNIVERSITE PARIS-EST CRETEIL VAL DE MARNE, CRETEIL (FR); UNIVERSITE PARIS-SUD 11, ORSAY CEDEX (FR)

(21) Appl. No.: 13/389,488

(22) PCT Filed: Aug. 9, 2010
(86) PCT No.: PCT/EP10/61566

§ 371 Date: Jul. 31, 2012

Related U.S. Application Data
(60) Provisional application No. 61/232,496, filed on Aug. 10, 2009.

(51) Int. Cl. C40B 30/04; A61P 35/00; A61K 51/00; C12Q 1/68; C40B 40/06

(52) U.S. Cl. 424/1.11; 435/6.11; 506/9; 435/6.12; 506/16

(57) ABSTRACT

The present invention provides a method for determining the RES phenotype in a tumor. The present invention further provides a method for predicting the sensitivity of a tumor to an epigenetic treatment, the method comprising determining the RES phenotype in said tumor, the presence of the RES phenotype in a tumor being indicative of a tumor sensitive to an epigenetic therapy. The present invention also provides a method for diagnosing an aggressive tumor and for selecting a patient affected with a tumor for an epigenetic therapy.

### FIELD OF THE INVENTION

The present invention relates to the field of medicine, in particular of oncology. It provides a new method for diagnosing an aggressive tumor and for predicting the sensitivity of a tumor to an epigenetic treatment.

### BACKGROUND OF THE INVENTION

Bladder cancer is the fifth cancer in term of incidence. It can appear as superficial lesions restricted to the urothelium (Ta and carcinoma in situ (CIS)) or to the lamina propria (T1) or as muscle invasive lesions (T2-T4). Two different pathways of tumour progression have been so far described in bladder cancer, the Ta pathway and the CIS pathway. Ta tumours which constitute 50% of bladder tumours at first presentation are superficial papillary tumour usually of low grade which do not invade the basal membrane. Carcinoma-in-situ (CIS) are also superficial tumour which do not invade the basal membrane but are always of high grade.

Ta tumours, despite chirurgical resection associated or not with BCG (Bacillus Calmette-Guerin) therapy, often recur but rarely progress to muscle invasive disease (T2-T4), whereas CIS often progress to T2-T4 tumors. Concerning muscle invasive bladder carcinomas, the standard treatment is cystectomy associated with chemotherapy and/or radiotherapy. Despite this radical treatment, muscle invasive bladder carcinoma remains a deadly disease for most patients.

Accordingly, there is a strong need for an appropriate treatment for bladder tumor of the CIS pathway, in particular for more effective therapeutic protocols.

Moreover, considering that most of anticancer treatments not only cause severe side effects but also are generally physically exhausting for patients and often associated with high costs, the choice of the appropriate therapeutic protocols is of capital importance.

Consequently, practitioners need methods for predicting the sensitivity of a tumor to a particular treatment prior to the actual onset of said treatment.

In a cancer cell, genetic and epigenetic lesions contribute to transcriptional deregulation. Genetic alterations associated with cancer, such as gene mutation, gene amplification, loss of heterozygosity or deletion, may affect single gene or extent to a whole region. Epigenetic changes include alteration of the genomic DNA methylation and histone modification profile. Until recently, epigenetic silencing in cancer has always been envisaged as a local event silencing discrete genes. However, recent findings indicate that large regions of chromosomes can be co-ordinately suppressed, with similar implication as loss of heterozygosity. This phenomenon has been named as long-range epigenetic silencing (LRES).

The mechanism of gene silencing within these regions may be due to DNA and histone modification or histone modification with no associated DNA methylation.

DNA methylation in mammals occurs mainly at cytosine residues in CpG dinucleotide pairs. Short stretches of CpG-dense DNA, known as CpG islands, are typically found associated with gene promoters. Most CpG island promoters are unmethylated, a state associated with active gene transcription. In contrast, CpG island promoters can become de novo methylated in a cancer cell and this methylation is associated with gene silencing.

Histones, in particular H3 and H4, have long tails protruding from the nucleosome which can be covalently modified. Well-described histone modifications include methylation, acetylation, phosphorylation, ubiquitination, sumoylation, citrullination, and ADP-ribosylation. Combinations of histone modifications result in different chromatine states and constitute a code, the so-called “histone code”. Typically, acetylation of histone tails is associated with active gene transcription whereas deacetylation is associated with silent gene. Methylation of lysine residues in histone H3 can have opposite effects, e.g. trimethylation of lysine 9 or 27 is associated with silent gene (Barski et al., 2007) whereas trimethylation of lysine 4 is associated with active gene transcription (Koch et al., 2007).

Long-range epigenetic silencing has been described in colon (Frigola et al., 2006) and breast cancers (Novak et al., 2006). These regions have been identified by detecting concordant methylation of adjacent CpG island gene promoters, followed by an examination of histone methylation.

### SUMMARY OF THE INVENTION

The inventors have herein demonstrated the existence of a particular regional epigenetic silencing (RES) phenotype which is present in tumors belonging to the more aggressive of the two pathways of bladder tumor progression, the carcinoma in situ pathway. Furthermore, the inventors have shown that tumors with this RES phenotype are particularly sensitive to epigenetic therapy.

Accordingly, the present invention concerns a method for determining the RES phenotype of a tumor, wherein the method comprises determining the expression level of at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and wherein the over-expression of said genes is indicative of the RES phenotype of the tumor. Optionally, the method further comprises determining the expression level of at least 3 genes selected from the group consisting of ANXA10, IGF2, B3GALNT1, EPHB6, SEMA6A, CXorf57, SLC15A1, HS6ST3 and KRT20, and wherein the absence of over-expression of said genes is indicative of the RES phenotype of the tumor or confirms its RES phenotype. Preferably, the method comprises determining the expression level of a first set of at least 24 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and a second set of at least 3 genes selected from the group consisting of ANXA10, IGF2, B3GALNT1, EPHB6, SEMA6A, CXorf57, SLC15A1, HS6ST3 and KRT20, and wherein the over-expression of the genes of the first set and the absence of over-expression of the genes of the second set is indicative of the RES phenotype of the tumor.

Alternatively, the present invention concerns a method for determining the RES phenotype of a tumor, wherein the method comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed and/or determining the number of chromosomal regions selected from the group consisting of regions 2-7, 3-2, 3-5, 7-2, 14-1, 19-3A and 19-3B which are silenced, and optionally assessing the expression level of the EZH2 histone methyltransferase in said tumor, and wherein the RES phenotype is defined either by the presence of at least three of said over-expressed genes and/or by the presence of at least three of said silenced regions, and/or by the presence of at least two of said silenced regions and an overexpression of the EZH2 histone methyltransferase. Preferably, the tumor is a bladder tumor.

In an embodiment, the method comprises determining the number of chromosomal regions selected from the group consisting of regions 2-7, 3-2, 3-5, 7-2, 14-1, 19-3A and 19-3B which are silenced in said tumor, and the RES phenotype is defined by the presence of at least three of said silenced regions.

In another embodiment, the method comprises determining the number of chromosomal regions selected from the group consisting of regions 2-7, 3-2, 3-5, 7-2, 14-1, 19-3A and 19-3B which are silenced, and assessing the expression level of the EZH2 histone methyltransferase in said tumor, and the RES phenotype is defined by the presence of at least two of said silenced regions and an overexpression of the EZH2 histone methyltransferase.

In a further embodiment, the method comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed, and the RES phenotype is defined by the presence of at least three of said over-expressed genes.

In a second aspect, the present invention concerns a method for diagnosing an aggressive tumor, wherein the method comprises determining the RES phenotype in a tumor with the method according to the invention, and wherein the presence of the RES phenotype in said tumor is indicative of an aggressive tumor. Preferably, the tumor is a bladder tumor. In an embodiment, the tumor belongs to the CIS pathway. In another embodiment, the tumor is a muscle-invasive or high grade tumor.

In a third aspect, the present invention concerns a method for predicting the sensitivity of a tumor to an epigenetic therapy, wherein the method comprises determining the RES phenotype in said tumor with the method according to the invention, and wherein the presence of the RES phenotype in said tumor is predictive that said tumor is sensitive to an epigenetic therapy. Preferably, the tumor is a bladder tumor.

In a further aspect, the present invention concerns a method for selecting a patient affected with a tumor for an epigenetic therapy or determining whether a patient affected with a tumor is susceptible to benefit from an epigenetic therapy, wherein the method comprises determining the RES phenotype of said tumor with the method according to the invention, and wherein the presence of the RES phenotype in said tumor is predictive that an epigenetic therapy is indicated for said patient. Preferably, the tumor is a bladder tumor.

In an embodiment, the epigenetic therapy comprises at least one compound selected from the group consisting of histone deacetylase inhibitors, histone methyltransferase inhibitors and histone demethylases, and any combination thereof. Preferably, the compound is an inhibitor of histone deacetylases HDAC1, HDAC2 and/or HDAC3, more preferably of HDAC1 and/or HDAC2.

In another embodiment, the epigenetic therapy further comprises at least one DNA methyltransferase inhibitor.

In another aspect, the present invention concerns an epigenetic compound for use in the treatment of cancer in a patient affected with a tumor with a RES phenotype. In an embodiment, the epigenetic compound is selected from the group consisting of histone deacetylase inhibitor, histone methyltransferase inhibitor and histone demethylase, and any combination thereof. In another embodiment, the epigenetic compound is used in combination with a DNA methyltransferase inhibitor. Preferably, the compound is an inhibitor of histone deacetylases HDAC1, HDAC2 and/or HDAC3, more preferably of HDAC1 and/or HDAC2. In a further embodiment, the epigenetic compound is used in combination with another antineoplastic agent.

In a last aspect, the present invention concerns a kit for determining the RES phenotype of a tumor, wherein the kit comprises detection means selected from the group consisting of a pair of primers, a probe and an antibody specific to a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2; or to b) the genes EZH2, CDC25B, TUBB3, CDH2, CXCL3, CXCL6, MLLT11, CXCL2, CTSL2, NFIL3, GPR161, CSRP2 and HDAC9; or a DNA chip for determining the RES phenotype of a tumor, wherein the DNA chip comprises a solid support which carries nucleic acids that are specific to a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2; or to b) the genes EZH2, CDC25B, TUBB3, CDH2, CXCL3, CXCL6, MLLT11, CXCL2, CTSL2, NFIL3, GPR161, CSRP2 and HDAC9.

### BRIEF DESCRIPTION OF THE DRAWINGS

FIG. 1: Identification of regions of downregulation independent of copy number changes (a) Identification of regions of correlated expression independent of copy number changes. Left panel: Transcriptome correlation map for region 7-2 based on the Affymetrix data for 57 bladder tumors. The significance threshold is indicated by a dashed green line (p<0.002) (Reyal et al. 2005). Three genes—SKAP2, HOXA1, HOXA5—have a significant transcriptome correlation score. Right panel: Transcriptome correlation map for the subset of tumors presenting no DNA copy number changes in region 7-2 (n=46). Five genes show correlated expression independent of copy number changes: SKAP2, HOXA1, HOXA2, HOXA4, HOXA5. (b) Comparison of Affymetrix and RT-qPCR data for regions 3-2 and 7-2. Upper panel: Affymetrix MASS data for regions 3-2 and 7-2. Lower panel: the mRNA levels of all the genes of these regions relative to 18S as determined by RT-qPCR in tumors T195, T259, T447 and T1207, in the cell line CL1207 and in normal samples (n=4) (lower panels). Each sample was studied by RT-qPCR in duplicate on a TLDA format. The histograms reflect the average value of the duplicates. For normal samples, the error bars indicate the standard deviation between four independent samples.

FIG. 2: Delineation of stretches of contiguously downregulated genes in the regions presenting a downregulation. RT-qPCR data led to the identification of stretches of contiguously downregulated genes in tumor T1207 and its derived cell line, CL1207, within nine of the ten regions presenting downregulation. Four normal samples were used for comparison. The stretches were defined as three or more consecutively downregulated genes in T1207 and CL1207 (ratio with average expression in normal samples <0.5). Genes that were not expressed were included in these stretches. The names of the genes, and the location and the size of the region are indicated.

FIG. 3: Effect of 5-aza-deoxycytidine, TSA or 5-aza-deoxycytidine+TSA on the expression of the genes located in the regions of downregulation. (a-d left panels) The cell line CL1207 was treated with 5-aza-deoxycytidine, TSA or 5-aza-deoxycytidine +TSA as described in Materials and Methods of the experimental section. The expression of genes located in the stretches of downregulation in regions 2-7, 3-2, 7-2 and 19-3A was measured by RT-qPCR on individual assays in the absence of (NT), or after treatment with 5-aza-deoxycytidine (5aza), TSA or 5-aza-deoxycytidine +TSA (5aza+TSA). Treatments were scored as having an effect when the ratio between treatment and non-treatment values was >1.5. (a-d right panels) NHU cells were treated with 5-aza-deoxycytidine +TSA. Results are expressed as the ratio of transcript expression in cells with or without treatment. For each treatment, RT-qPCR analyses were performed on two independent experiments, with each qPCR performed in duplicate. The error bars indicate the variation between the means of the two independent experiments.

FIG. 4: Effect of 5-aza-deoxycytidine, TSA or 5-aza-deoxycytidine +TSA on the expression of the genes located within the regions of downregulation. (a-e) The cell line CL1207 was treated with 5-azadeoxycytidine, TSA or 5-aza-deoxycytidine +TSA as described in Materials and Methods of the experimental section. The expression of the genes located within the stretches of downregulation in regions 3-5, 6-7, 14-1, 17-7 and 19-3B were measured by RT-qPCR in individual assays in the absence (NT) or after treatment with 5-aza-deoxycytidine (5aza), TSA or 5-aza-deoxycytidine +TSA (5aza +TSA). The treatments were scored as having an effect (resulting in re-expression) if the fold-change between treated and non-treated was greater than 1.5. For each treatment, RT-qPCR analyses were performed on two independent experiments, each measured in duplicate. The error bars indicate the variation between the means of the two independent experiments.

FIG. 5: Analyses of epigenetic modifications in regions 2-7 and 19-3A (a) Schematic map of region 2-7 (not to scale) with CpG islands, according to the UCSC browser. The number of CpG in each island is indicated. (b) DNA methylation analyses for region 2-7: bisulfite sequencing analyses for CpG 141 and CpG 39 covering a promoter region. In each case, T1207 DNA, CL1207 DNA, NHU DNA and SssI-methylated DNA (“Met. DNA”) were studied. Each row represents an individual clone, and each box (methylated in black, unmethylated in white) represents an individual CpG site. The curved arrows indicate the transcription start sites. (c) ChIP assays were performed for promoters in region 2-7 with antibodies against trimethyl H3K9 (left panel), trimethyl H3K27 (middle panel) and acetyl H3K9 (right panel). The bar chart shows the amount of immunoprecipitated target DNA expressed as a percentage of total input DNA, measured in duplicate by qPCR. The error bars indicate the variation between the means of two independent experiments. (d) Schematic map of region 19-3A with CpG islands. (e) ChIP analyses for region 19-3A as in (c).

FIG. 6: Analyses of epigenetic modifications in region 3-2. (a) Schematic map of region 3-2 (not to scale) with CpG islands, according to the UCSC browser. The number of CpGs in each island is indicated. (b) DNA methylation analyses for region 3-2: bisulfite sequencing analyses for the three CpGs covering a promoter region: CpG 74, 59 and 68. In each case, DNA from T1207, CL1207 and NHU and SssI-methylated DNA (“Met. DNA”) were studied. Each row represents an individual clone and each box represents an individual CpG site. A black box indicates methylation and a white box no methylation. The curved arrows indicate the transcription start sites. (c) Methylation analysis of DLEC1 promoter by bisulfite sequencing in five tumor samples showing downregulation of region 3-2: T195, T259, T447, T910 and T1448. (d) ChIP assays were performed on promoters in region 3-2 with antibodies against trimethyl H3K9 (left panel), trimethyl H3K27 (middle panel) and acetyl H3K9 (right panel). The bar chart shows the amount of immunoprecipitated target DNA expressed as a percentage of total input DNA, measured in duplicate by qPCR. The error bars indicate the variation between the means of two independent experiments.

FIG. 7: Analyses of epigenetic modifications in regions 3-5, 7-2, 14-1 and 19-3B (a) Methylation analysis of the promoter-associated CpG island of HOXA5 by COBRA. A fragment of the promoter was amplified and digested with MboI; if the PCR product is digested, the studied CpG site is methylated (see Material and Methods in experimental section). T1207 and CL1207 did not show the same methylation pattern, even if the genes were down-regulated in both samples compared to normal urothelium. (b) Analysis of H3K9 and H3K27 trimethylation, and H3K9 acetylation for one gene in each of four down-regulated regions (3-5, 7-2, 14-1 and 19-3B) in CL1207 before and after treatment by trichostatin A (TSA) and in NHU cells. The bar chart shows the amount of immunoprecipitated target DNA expressed as a percentage of total input DNA, measured in duplicate by qPCR. The error bars indicate the variation between the means of two independent experiments.

FIG. 8: Identification of a multiple regional epigenetic silencing phenotype. (a) Summary of the individual cluster analysis for each region (obtained from FIG. 1). Each row represents a region, and each column a tumor or normal sample. If a region in a sample is downregulated, the corresponding box is colored. Twenty-three tumors (below the horizontal gray line) displayed downregulation of at least 3 regions. The position of tumor T1207 is indicated by an arrow. (b) Cluster analysis of 57 tumor samples and 5 normal samples: samples are clustered according to their region expression score, which corresponds to the average downregulation in each region (see Methods). Tumors displaying downregulation of several regions (below the horizontal green line) define a regional epigenetic silencing (RES) phenotype. All samples are annotated (indicated by triangles below the figure) with their stage and grade, their carcinoma in situ signature and their FGFR3 mutation status. The classification obtained from FIG. 8a is also indicated (i.e. tumors displaying at least three downregulated regions in FIG. 8a). The cluster analysis was not affected by the exclusion of region expression scores for tumors displaying genetic loss in the corresponding region (data not shown). (c) In bladder cancer, two different pathways can lead to invasive tumors: the superficial Ta tumor pathway, in which progression is rare, and the carcinoma in situ (CIS) pathway, in which the superficial lesions (CIS) are high-grade and very often progress to T1 and T2-T4 tumors. The percentages of FGFR3 mutations at the different stages of tumor progression in the two pathways are taken from Saison-Behmoaras et al., (1991). (d) EZH2 mRNA quantification in all tumor and normal samples measured by RT-qPCR in duplicate. Samples are in the same order as in 8b. Normal samples are indicated by red dots. The tumors with the RES phenotype, as defined in FIG. 8c, are surrounded by a rectangle.

FIG. 9: CIS signature and RES phenotype for an independent bladder tumor set (n=40) Cluster analysis of the group of 40 tumor samples and 3 normal samples: samples are clustered according to their region expression score, which corresponds to the average downregulation in each region (see Methods in experimental section). For this additional set, expression of genes in the 7 stretches of downregulation defined in FIG. 2 was measured using TLDA and used to calculate the region expression score. Tumors displaying downregulation of several regions (below the horizontal gray line) defined a regional epigenetic silencing (RES) phenotype. All samples are annotated (indicated by triangles below the figure) according to their stage and grade, their carcinoma in situ signature (Dyrskjot et al., 2004) and their FGFR3 mutation status.

FIG. 10: Characterization of the regional epigenetic phenotype in bladder cancer cell lines. (a) Comparison of mRNA expression for genes in region 2-7 before and after treatment with TSA in bladder cancer cell lines and NHU cells; results obtained for the CL1207 cells (from FIG. 3) are also shown. Transcript values were measured by RT-qPCR using TLDA (see Materials and Methods in experimental section). The ratio between treated and non-treated cells is shown. Error bars represent the variation between the means of two independent experiments of TSA treatment. (b) Same comparison for region 3-2. (c) Same comparison for region 19-3A. (d) Summary of the effects of TSA treatment on all bladder cancer and NHU cell lines studied. Only groups of genes in which at least two genes were re-expressed after treatment are considered (see FIG. 11). (e) ChIP analysis in TCCSUP and RT112 cells for regions 2-7, 3-2 and 19-3A, with an antibody against trimethyl residues on lysine 9 of histone 3. The bar chart shows the amount of immunoprecipitated target DNA expressed as a percentage of total input DNA, measured in duplicate by qPCR. The error bars indicate the variation between the means of two independent experiments.

FIG. 11: Comparison of the mRNA levels of the genes in regions 3-5, 7-2, 14-1 and 19-3B in bladder cancer and normal cells before and after treatment with TSA. mRNA levels were assessed by RT-qPCR using TLDA (see Materials and Methods in experimental section). The ratio between treated versus non-treated cells is shown. For the sake of clarity, results obtained for the CL1207 cells (presented in FIG. 3) were also indicated. Error bars represent the variation between two independent experiments. For each region and cell line, groups of contiguous genes (n≧2) that were re-expressed (fold-change>1.5) in cancer cell lines are identified and used to define a specific regional epigenetic alteration, as reported in FIG. 10d.

FIG. 12: Histone methylation and acetylation studies in TCCSUP and RT112 cells (a) ChIP analysis in TCCSUP and RT112 cells for region 2-7 using antibodies against trimethyl residues on lysine 27 of histone 3 and acetyl residue on lysine 9 of histone 3. The bar chart shows the amount of immunoprecipitated target DNA expressed as a percentage of total input DNA, measured in duplicate by qPCR. The error bars indicates the variation between the means of two independent experiments. (b) Similar experiments for region 3-2. (c) Similar experiments for region 19-3A. (d) Analysis of H3K9 and H3K27 tri-methylation, and H3K9 acetylation for one gene in each of four down-regulated regions (3-5, 7-2, 14-1 and 19-3B) in TCCSUP and RT112 cells.

FIG. 13: Cell viability after treatment with various doses of trichostatin A in bladder cancer cell lines with or without the RES phenotype and NHU cells. The percentage of surviving treated versus non-treated cells as a function of TSA concentration for various bladder cancer (MGHU3, RT112, T24, TCCSUP, HT1376, JMSU1, CL1207) and normal (NHU) cell lines is indicated. The number of cells surviving post treatment with TSA for 72 hours was counted and compared to control (no treatment) cultures. Cell lines with the RES phenotype are indicated with full symbols (TCCSUP, HT1376, JMSU1, CL1207) whereas cell lines without the RES phenotype (NHU, MGHU3, RT112, T24) are indicated with empty symbols. The error bars indicate the mean variation between two independent experiments.

FIG. 14: Expression of the gene markers of the RES phenotype in tumor samples and normal tissue samples. The expression of EZH2, CDC25B, TUBB3, CDH2, CXCL3, CXCL6, MLLT11, CXCL2, CTSL2, NFIL3, GPR161 and CSRP2 genes was assessed for each sample. Grey box indicate that the gene is over-expressed in said sample. The RES phenotype is specified for each sample: +: presence of the RES phenotype; −: absence of the RES phenotype. The type of the sample is indicated in the fourth column: T: tumor; NHU: normal human urethelium; M: muscle. Tumors belonging to the CIS pathway are indicated in the fifth column by a + sign. The stade and the grade of each tumor sample are also indicated.

FIG. 15: HDAC9 expression level in invasive bladder tumors with/without RES phenotype. HDAC9 log 2 mRNA expression level according to Affymetrix U133plus2 arrays in normal samples (n=4), invasive tumors without the RES phenotype (n=29) and invasive tumors with the RES phenotype (n=74). P-value obtained from a two-tailed t-test between tumors with and without RES phenotype are indicated.

FIG. 16: EZH2 trimethyltransferase in invasive bladder tumors and bladder cancer cell lines with/without the RES phenotype. (a) EZH2 log 2 mRNA expression level according to Affymetrix U133plus2 arrays in normal samples (n=4), invasive tumors without the regional epigenetic silencing phenotype (n=29) and invasive tumors with the RES phenotype (n=74). P-value obtained from a two-tailed ttest between tumors with and without RES phenotype are indicated. (b) Validation of Affymetrix expression data by RT-qPCR analysis using a TLDA format for 40 tumor samples. (c) EZH2 mRNA expression levels in cell lines with (n=4) and without (n=3) RES phenotype. The expression level in normal human urothelial (NHU) is shown for comparison.

FIG. 17: The knockout of EZH2 reverses the regional epigenetic alteration in chromosomal regions 2-7 and 3-2. (a) ChIP assays were performed for promoters and within genes in regions 2-7 (left panel) and 3-2 (right panel) with an antibody against H3K27me3 in CL1207 cells, which display a RES phenotype in regions 2-7 and 3-2. The bar chart shows the amount of immunoprecipitated target DNA expressed as a percentage of total input DNA, measured in duplicate by qPCR. The error bars indicate the variation between the means of two independent experiments. (b) Left panel: mRNA expression levels in region 2-7 before and after siRNA experiment. Right panel: same analysis in region 3-2. (c) Left panel: ChIP assays performed for promoters in region 2-7 with an antibody against H3K27me3 before and after transfection with EZH2 targeted siRNA. Right panel: same analysis in region 3-2.

FIG. 18: Effect of MS275 on the expression of the genes located in the regions of downregulation. The cell line CL1207 was treated with MS275, or TSA as described in Materials and Methods of the experimental section. The expression of genes located in the stretches of downregulation in regions 2-7 and 3-2 was measured by RT-qPCR on individual assays in the absence of (NT), or after treatment with MS275 or TSA. Treatments were scored as having an effect when the ratio between treatment and non-treatment values was >1.5. Results are expressed as the ratio of transcript expression in cells without treatment.

FIG. 19: Number of classification errors of RES phenotype according to the number of genes.

### DETAILED DESCRIPTION OF THE INVENTION

A large scale bioinformatics analysis combining paired transcriptome and comparative genomic hybridization (CGH) array data was used to identify regions of neighbouring genes with correlated expression patterns that were not dependent upon changes in copy number. When applied to a series of bladder cancers, this approach led to identify 28 regions of correlated expression that were recognized as candidate regions controlled by epigenetic mechanisms (Stransky et al. 2006).

The inventors have herein demonstrated that some of these regions are silenced by epigenetic alterations involving histone modifications with very rare CpG promoter DNA methylation. They have further showed the existence of a regional epigenetic silencing (RES) phenotype as these particular silenced regions are simultaneously silenced in the same subset of tumors. Strikingly, this subset of tumors belongs to the more aggressive of the two pathways of bladder tumor progression, the carcinoma in situ pathway. Furthermore, in studies herein described, inventor's data reveal that tumors with this RES phenotype are particularly sensitive to epigenetic therapy.

### DEFINITIONS

The term “epigenetic compound” as used herein refers to a compound that is able to reverse epigenetic aberrations. An epigenetic compound may be a histone deacetylase inhibitor, a histone methyltransferase inhibitor, a histone demethylase or a DNA methyltransferase inhibitor. Preferably, the epigenetic compound is a histone deacetylase inhibitor, a histone methyltransferase inhibitor or a histone demethylase. More preferably, the epigenetic compound is a histone deacetylase inhibitor and/or a histone methyltransferase inhibitor.

The term “histone deacetylase inhibitor” refers to a compound that interferes with the function of at least one histone deacetylase. A histone deacetylase is a protein that catalyzes removal of an acetyl group from the epsilon-amino group of lysine side chains in histones (H2A, H2B, H3 or H4), thereby reconstituting a positive charge on the lysine side chain and leading to the formation of a condensed and transcriptionally silenced chromatin. In an embodiment, the histone deacetylase inhibitor is selected from the group consisting of a peptide, an antibody, an antigen binding fragment of an antibody, a nucleic acid, an aliphatic acid, a hydroxamic acid, a benzamide, depudecin, and an electrophilic ketone, and a combination thereof. In a particular embodiment, the histone deacetylase inhibitor is an oligonucleotide that inhibits expression or function of histone deacetylase, such as an antisense molecule or a ribozyme. Alternatively, the histone deacetylase inhibitor is a dominant negative fragment or variant of histone deacetylase. Examples of histone deacetylase inhibitors include, but are not limited to, trichostatin A, vorinostat (suberoylanilide hydroxamic acid or SAHA), valproic acid, belinostat (PXD101), Panobinostat (LBH-589), MS-275, N-acetyldinaline (CI-994), depudecin, oxamflatin, bishydroxyamic acid, MGCD0103, Scriptaid, apicidin, derivatives of apicidin, benzamide, derivatives of benzamide, FR901228, FK228, trapoxin A, trapoxin B, HC-toxin, chlamydocin, Cly-2, WF-3161, Tan-1746, pyroxamide, NVP-LAQ824, butyrate, phenylbutyrate, hydroxyamic acid derivatives, cyclic hydroxamic acid-containing peptide (CHAP), m-carboxycinnamic acid bishydroxamic acid (CBHA), suberic bishydroxyamic acid and azelaic bishydroxyamic acid, and a salt thereof. In a particular embodiment, the histone deacetylase inhibitor is selected from the group consisting of trichostatin A, vorinostat, valproic acid, panobinostat and belinostat. In a preferred embodiment, the histone deacetylase inhibitor is vorinostat. More preferably, the compound is an inhibitor of histone deacetylases HDAC1, HDAC2 and/or HDAC3, more preferably of HDAC1 and/or HDAC2. Still more preferably, the compound has specificity for the HDAC of class I, in particular for the HDAC1, HDAC2 and/or HDAC3, preferably HDAC1 and/or HDAC2. In particular, the inhibitor may be MS-275 or SK-7041, SK-7068, Pyroxamide, Apicidin, Depsipeptides, MGCD-0103, Depudecin.

The term “histone methyltransferase inhibitor” refers to a compound that interferes with the function of at least one histone methyltransferase. A histone methyltransferase is a histone-lysine N-methyltransferase (registry number EC 2.1.1.43) or a histone-arginine N-methyltransferase (registry number EC 2.1.1.23). These enzymes catalyze the transfer of one to three methyl groups from the cofactor S-Adenosyl methionine to lysine or arginine residues of histone proteins. In an embodiment, the histone methyltransferase inhibitor is selected from the group consisting of a peptide, an antibody, an antigen binding fragment of an antibody, a nucleic acid and a drug, and a combination thereof. In a particular embodiment, the histone methyltransferase inhibitor is an oligonucleotide that inhibits expression or function of histone methyltransferase, such as an antisense molecule or a ribozyme. Alternatively, the histone methyltransferase inhibitor is a dominant negative fragment or variant of histone methyltransferase. In a particular embodiment, the histone methyltransferase inhibitor inhibits a histone methyltransferase selected from the group consisting of EZH2, G9A, ESET, SUV39h1, SUV39h2 and Eu-HMTase1. In a particular embodiment, the histone methyltransferase inhibitor is selected from the group consisting of BIX-01294 (Kubicek et al., 2007), Chaetocin (Greiner et al., 2005) and 3-Deazaneplanocin A. In another particular embodiment, the histone methyltransferase inhibitor is a siRNA which specifically inhibits the expression of EZH2.

The term “histone demethylase” refers to proteins which are able to reverse histone methylation. Examples of histone demethylases include JMJD2 family of proteins (Whetstine et al., 2006), in particular JMJD2C, JMJD3, JMJD1A, JHDM3 family and JMJD3/UTX proteins. In particular, proteins of the JHDM1 family include JHDM1A, proteins of the JHDM3/JMJD2 subfamily include JMJD2A/JHDM3A, JMJD2B, JMJD2C/GASC1 and JMJD2D, proteins of the JARID subfamily include JARID1A, JARID B, JARID C and JARID D, proteins of the UTX/UTY sub-family include UTX and JMJD3, proteins of the JHDM2 subfamily include JHDM2A, JHDM2B and JHDM2C. The histone demethylase may further include the peptidyl arginine deiminase PADI4 or the flavin-dependent amine oxidase LSD1. In a preferred embodiment, the histone demethylase is able to reverse H3K9me3 and/or H3K27me3 histone modification.

The common nomenclature of histone modifications is as follows: first, the name of the histone (e.g H3), second the single letter amino acid abbreviation (e.g. K for Lysine) and the amino acid position in the protein, and third the type of modification (Me: methyl, P: phosphate, Ac: acetyl, Ub: ubiquitin). As example, H3K9me3 denotes the trimethylation of the 9th residue (a lysine) from the N-terminal of the H3 protein and H3K9ac denotes the acetylation of the 9th residue (a lysine) from the N-terminal of the H3 protein.

The term “DNA methyltransferase inhibitor” refers to a compound that interferes with the function of at least one DNA methyltransferase. A DNA methyltransferase (DNMT) is an enzyme that catalyzes the transfer of a methyl group to DNA. Four active DNA methyltransferases have been identified in mammals, namely DNMT1, DNMT2, DNMT3A and DNMT3B. The DNA methyltransferase inhibitor may be selected from the group consisting of a peptide, an antibody, an antigen binding fragment of an antibody, a nucleic acid and a drug, and a combination thereof. In a particular embodiment, the DNA methyltransferase inhibitor is an oligonucleotide that inhibits expression or function of DNA methyltransferase, such as an antisense molecule or a ribozyme. Alternatively, the DNA methyltransferase inhibitor is a dominant negative fragment or variant of DNA methyltransferase. Examples of DNA methyltransferase inhibitors include, but are not limited to, 5-azacytidine (5-azaCR), decitabine (5-aza-2′-deoxycytidine or 5-aza-CdR), 5-fluoro-2′-deoxycytidine, 5,6-dihydro-5-azacytidine, procaine, (−)-epigallocatechin-3-gallate (EGCG), zebularine (1-(beta-d-ribofuranosyl)-1,2-dihydropyrimidin-2-one), NSC 303530 (Siedlecki et al., J Med. Chem. 2006, 49(2):678-83), NSC 401077 (RG108), procainamide, hydralazine, psammaplin A and MG98. Other examples include compounds described in patent applications WO 2008/033744, WO 99/12027, WO 2005/085196, EP 1 844 062 and WO 2006/060382, and in the article of Siedlecki et al. (Siedlecki et al., 2006).

The term “epigenetic therapy” as used herein refers to a treatment involving at least one epigenetic compound. In an embodiment, an “epigenetic treatment” or “epigenetic therapy” refers to a treatment involving at least a histone deacetylase inhibitor, a histone methyltransferase inhibitor and/or a histone demethylase, preferably involving at least a histone deacetylase inhibitor. In a preferred embodiment, an epigenetic treatment refers to a treatment involving at least one histone deacetylase inhibitor and at least one histone methyltransferase inhibitor. In a particular embodiment, an epigenetic treatment refers to a treatment involving at least a histone deacetylase inhibitor, a histone methyltransferase inhibitor and/or a histone demethylase, in combination with a DNA methyltransferase inhibitor.

The term “cancer” or “tumor” as used herein refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). Typical cancers are breast, stomach, oesophageal, sarcoma, ovarian, endometrium, bladder, cervix uteri, rectum, colon, lung or ORL cancer, paediatric tumours (neuroblastoma, glyoblastoma multiforme), lymphoma, leukaemia, myeloma, seminoma, Hodgkin and malignant hemopathies. Preferably, the cancer is a solid cancer. More preferably, the cancer is selected from the group consisting of bladder cancer, colorectal cancer, oesophageal cancer, neuroblastoma, breast cancer and lung cancer. Even more preferably, the cancer is selected from the group consisting of bladder cancer, colorectal cancer and breast cancer. Even more preferably, the cancer is a bladder cancer. In a particular embodiment, the cancer is an epithelial-derived cancer.

Based on the microscopic appearance of cancer cells, pathologists commonly describe tumor grade by four degrees of severity: Grades 1, 2, 3, and 4. The cells of Grade 1 tumors resemble normal cells, and tend to grow and multiply slowly. Conversely, the cells of Grade 3 or Grade 4 tumors do not look like normal cells of the same type. Grade 3 and 4 tumors tend to grow rapidly and spread faster than tumors with a lower grade. Usually, tumors are grading as follow: G1: Well-differentiated (Low grade); G2: Moderately differentiated (Intermediate grade); G3: Poorly differentiated (High grade); and G4: Undifferentiated (High grade). As used herein, a high grade tumor is a tumor of G3 or G4 grade.

By “bladder tumor” is intended herein urinary bladder tumor, bladder cancer, bladder carcinoma or urinary bladder cancer, and bladder neoplasm or urinary bladder neoplasm. A bladder tumor can be a bladder carcinoma or a bladder adenoma. The most common staging system for bladder tumors is the TNM (tumor, node, metastasis) system. This staging system takes into account how deep the tumor has grown into the bladder, whether there is cancer in the lymph nodes and whether the cancer has spread to any other part of the body. The following stages are used to classify the location, size, and spread of the cancer, according to the TNM staging system: Stage 0 (CIS or Ta): Cancer cells are found only on the inner lining of the bladder; Stage I (T1): Cancer cells have started to grow into the connective tissue beneath the bladder lining; Stage II (T2): Cancer cells have grown through the connective tissue into the muscle; Stage III (T3): Cancer cells have grown through the muscle into the fat layer; Stage IV (T4): Cancer cells have proliferated to the lymph nodes, pelvic or abdominal wall, and/or other organs. In an embodiment, the bladder tumor is a bladder carcinoma. In a particular embodiment, the bladder tumor belongs to the carcinoma in situ (CIS) pathway. In another particular embodiment, the bladder tumor is a muscle-invasive tumor, i.e. T2-T4 tumor or a high grade tumor (G3 or G4). As used herein, the term “aggressive bladder tumor” refers to a high-grade (G3 or G4) tumor, T2-T4 tumors and tumors of the CIS pathway. Preferably, the term “aggressive bladder tumor” refers to tumors of the CIS pathway.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease. In particular, the term “to treat a cancer”, “treating a cancer”, “to treat a tumor” or “treating a tumor” means reversing, alleviating, inhibiting the progress of, or preventing, either partially or completely, the growth of tumors, tumor metastases, or other cancer-causing or neoplastic cells in a patient.

As used herein, the term “subject” or “patient” refers to an animal, preferably to a mammal, even more preferably to a human, including adult, child and human at the prenatal stage. However, the term “subject” or “patient” can also refer to non-human animals, in particular mammals such as dogs, cats, horses, cows, pigs, sheeps and non-human primates, among others, that are in need of treatment.

The term “sample”, as used herein, means any sample containing cells derived from a subject, preferably a sample which contains nucleic acids. Examples of such samples include fluids such as blood, plasma, saliva, urine and seminal fluid samples as well as biopsies, organs, tissues or cell samples. The sample may be treated prior to its use, e.g. in order to render nucleic acids available. The term “cancer sample” or “tumor sample” refers to any sample containing tumoral cells derived from a patient, preferably a sample which contains nucleic acids. Preferably, the sample contains only tumoral cells. The term “normal sample” refers to any sample which does not contain any tumoral cell.

The methods of the invention as disclosed below, may be in vivo, ex vivo or in vitro methods, preferably in vitro methods.

In a first aspect, the present invention concerns a method for identifying chromosomal regions which could be involved in the RES phenotype of a given type of tumors, said method comprising: (a) identifying chromosomal regions with correlated expression; (b) excluding tumors with copy-number alteration; (c) selecting regions presented downregulation; (d) selecting regions containing at least 3 downregulated or non expressed contiguous genes; and (e) selecting regions silenced by histone modification.

In steps (a) and (b), copy number-independent regions of correlated expression are identified by combining transcriptome and CGH array data for a set of tumors belonging to a type of tumors of interest. For example, the identification of such chromosomal regions has been described for a set of bladder tumors in the article of Stransky et al. (Stransky et al., 2008; the disclosure of which is incorporated herein by reference). In summary, a transcriptome correlation map (TCM) which assesses the correlation which exists between the expression of a gene and those of neighbors is established (step (a)). CGH array analyses of the same set of tumors lead to identification of tumors that show genetic losses or gains. A new TCM is then recalculated, with exclusion of these tumors with copy-number alterations, and chromosomal regions with copy number-independent are identified (step (b)).

In step (c), regions with correlated expression due to down-regulation are selected among regions selected in step (b). For each correlated gene, the ratio between its expression value in each tumor sample and its mean expression in normal samples is calculated. These expression ratios are then used to cluster, for each region, all normal and tumor samples. For selected regions, the deregulation is represented by all or a subset of tumors. Preferably, at least three normal samples are used, more preferably at least five.

In step (d), regions containing a stretch of downregulated or non-expressed genes are selected among regions selected in step (c).

Finally, in step (e), regions silenced by histone modifications are selected among regions selected in step (d). These regions comprise very rare methylated promoter and thus DNA methylation is not significant enough to explain the silencing of these regions.

These regions are identified based on the study of a set of tumors of the same type but of varying grade and stage. Preferably, the set comprises at least 20 tumors. More preferably, the set comprises at least 50 tumors.

This method may be applied on sets of tumors of any type of cancer and chromosomal regions which could be involved in the RES phenotype in said cancer may be thus identified. Based on a set of bladder tumors, the chromosomal regions implicated in the RES phenotype in bladder cancer have been identified. These regions are regions 2-7, 3-2, 3-5, 7-2, 14-1, 19-3A and 19-3B.

The present invention concerns a method for determining the RES phenotype of a tumor, wherein the method comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed and/or determining the number of chromosomal regions selected from the group consisting of regions 2-7, 3-2, 3-5, 7-2, 14-1, 19-3A and 19-3B which are silenced, and optionally assessing the expression level of the EZH2 histone methyltransferase in said tumor, and wherein the RES phenotype is defined either by the presence of at least three of said over-expressed genes and/or by the presence of at least three of said silenced regions, and/or by the presence of at least two of said silenced regions and an overexpression of the EZH2 histone methyltransferase.

In an embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

In an embodiment, the method further comprises the step of providing a tumor sample from a subject.

Generally, the expression level of a gene is determined as a relative expression level. More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a particular embodiment, gene expression is determined by measuring the quantity of mRNA. For example the nucleic acid contained in the sample (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA). Amplification primers may be easily designed by the skilled person.

In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art.

Gene expression in samples may be normalized by using expression levels of proteins which are known to have stable expression such as RPLPO (acidic ribosomal phosphoprotein PO), TBP (TATA box binding protein), GAPDH (glyceraldehyde 3-phosphate dehydrogenase), β-actin or 18rRNA.

Gene expression levels in tumor sample are then compared with gene expression levels in normal sample. Preferably, the normal sample is provided from the same tissue type than the tumor sample. In an embodiment, the tumor sample is a sample of bladder tumor and the normal sample is a sample of normal urothelium. The normal sample may be obtained from the subject affected with the cancer or from another subject, preferably a normal or healthy subject, i.e. a subject who does not suffer from a cancer.

A gene is considered as silenced in tumor sample if, after normalization, the expression level of this gene is at least 1.5-fold lower than its expression level in the normal sample. Preferably, a gene is considered as silenced in tumor sample if, after normalization, the expression level of this gene is at least 2, 3, 4 or 5-fold lower than its expression level in the normal sample.

A gene is considered as over-expressed in tumor sample if, after normalization, the expression level of this gene is at least 1.5-fold higher than its expression level in the normal sample. Preferably, a gene is considered as over-expressed in tumor sample if, after normalization, the expression level of this gene is at least 2, 3, 4, or 5-fold higher than its expression level in the normal sample. In a preferred embodiment, a gene is considered as over-expressed in a tumor sample if, after normalization, the expression level of this gene is at least 2-fold higher than its expression level in the normal sample.

In an embodiment, the method for determining the RES phenotype of a tumor comprises determining the number of chromosomal regions selected from the group consisting of regions 2-7, 3-2, 3-5, 7-2, 14-1, 19-3A and 19-3B which are silenced in said tumor, wherein the RES phenotype is defined by the presence of at least three of said silenced regions.

Chromosomal regions are identified according to the International System for Human Cytogenetic Nomenclature (ISCN) fixed by the Standing Committee on Human Cytogenetic Nomenclature. Short arm locations are labeled p and long arms q. Each chromosome arm is divided into regions labeled p1, p2, p3 etc., and q1, q2, q3, etc., counting outwards from the centromere. Regions are delimited by specific landmarks, which are consistent and distinct morphological features, such as the ends of the chromosome arms, the centromere and certain bands. Regions are divided into bands labeled p11, p12, p13, etc., sub-bands labeled p11.1, p11.2, etc., and sub-sub-bands e.g. p11.21, p11.22, etc., in each case counting outwards from the centromere.

The region 2-7 is considered as silenced if at least three contiguous genes comprised in this region and selected from the group consisting of HOXD4, HOXD3, HOXD1 and MTX2 genes are silenced. These genes are located on chromosome 2 in location 2q31. In an embodiment, HOXD4, HOXD3 and HOXD1 are silenced. In another embodiment, HOXD3, HOXD1 and MTX2 are silenced. In a preferred embodiment, HOXD4, HOXD3, HOXD1 and MTX2 are silenced.

The region 3-2 is considered as silenced if at least three contiguous genes comprised in this region and selected from the group consisting of VILL, PLCD1, DLEC1 and ACAA1 genes are silenced. These genes are located on chromosome 3 in location 3p22-p21.3. In an embodiment, VILL, PLCD1 and DLEC1 are silenced. In another embodiment, PLCD1, DLEC1 and ACAA1 are silenced. In a preferred embodiment, VILL, PLCD1, DLEC1 and ACAA1 are silenced.

The region 3-5 is considered as silenced if at least three contiguous genes comprised in this region and selected from the group consisting of TCTA, AMT, NICN1, DAG1, BSN, APEH, RNF123 and GMPPB genes are silenced. These genes are located on chromosome 3 in location 3p21-24.3. In an embodiment, TCTA, AMT and NICN1 are silenced. In another embodiment, AMT, NICN1 and DA G are silenced. In another embodiment, NICN1, DA G and BSN are silenced. In a further embodiment, DAG1, BSN and APEH are silenced. In another embodiment, BSN, APEH and RNF123 are silenced. In a further embodiment, APEH, RNF123 and GMPPB are silenced. In a preferred embodiment, TCTA, AMT, NICN1, DAG1, BSN, APEH, RNF123 and GMPPB are silenced.

The region 7-2 is considered as silenced if at least three contiguous genes comprised in this region and selected from the group consisting of SKAP2, HOXA1, HOXA2, HOXA3, HOXA4 and HOXA5 genes are silenced. These genes are located on chromosome 7 in location 7p15. In an embodiment, SKAP2, HOXA1 and HOXA2 are silenced. In another embodiment, HOXA1, HOXA2 and HOXA3 are silenced. In another embodiment, HOXA2, HOXA3 and HOXA4 are silenced. In a further embodiment, HOXA3, HOXA4 and HOXA5 are silenced. In a preferred embodiment, SKAP2, HOXA1, HOXA2, HOXA3, HOXA4 and HOXA5 are silenced.

The region 14-1 is considered as silenced if at least three contiguous genes comprised in this region and selected from the group consisting of CMTM5, MYH6, MYH7, THTPA, AP1G2, DHRS2 and DHRS4 genes are silenced. These genes are located on chromosome 14 in location 14q1-12. In an embodiment, CMTM5, MYH6 and MYH7 are silenced. In another embodiment, THTPA, AP1G2 and DHRS2 are silenced. In a further embodiment, AP1G2, DHRS2 and DHRS4 are silenced. In a preferred embodiment, CMTM5, MYH6, MYH7, THTPA, AP1G2, DHRS2 and DHRS4 are silenced.

The region 19-3A is considered as silenced if at least three contiguous genes comprised in this region and selected from the group consisting of CYP4F3, CYP4F12, CYP4F2 and CYP4F11 genes are silenced. These genes are located on chromosome 19 in location 19p13. In an embodiment, CYP4F3, CYP4F12 and CYP4F2 are silenced. In another embodiment, CYP4F12, CYP4F2 and CYP4F11 are silenced. In a preferred embodiment, CYP4F3, CYP4F12, CYP4F2 and CYP4F11 are silenced.

The region 19-3B is considered as silenced if at least B3GNT3, INSL3 and JAK3 genes comprised in this region are silenced. These genes are located on chromosome 19 in location 19p13.

In an embodiment, the RES phenotype is defined by the presence of at least 3 of the silenced chromosomal regions described above. In another embodiment, the RES phenotype is defined by the presence of at least 4 of said regions. In a further embodiment, the RES phenotype is defined by the presence of at least 5 of said regions.

In another embodiment, the method for determining the RES phenotype of a tumor comprises determining the number of chromosomal regions selected from the group consisting of regions 2-7, 3-2, 3-5, 7-2, 14-1, 19-3A and 19-3B which are silenced, and assessing the expression level of the EZH2 histone methyltransferase in said tumor, wherein the RES phenotype is defined by the presence of at least two of said silenced regions and an overexpression of the EZH2 histone methyltransferase.

The number of chromosomal regions selected from the group consisting of regions 2-7,3-2, 3-5, 7-2, 14-1, 19-3A and 19-3B which are silenced, may be assessed as described above.

EZH2 is the catalytic subunit of Polycomb repressive complex 2 (PRC2), which is a highly conserved histone methyltransferase that targets lysine-27 of histone H3. The expression of this enzyme may be assessed by any method known by the skilled person such as quantitative or semi quantitative RT-PCR as well as real-time quantitative or semi quantitative RT-PCR, as described above.

In a particular embodiment, the RES phenotype is defined by the presence of at least three of silenced chromosomal regions selected from the group consisting of regions 2-7, 3-2, 3-5, 7-2, 14-1, 19-3A and 19-3B and an overexpression of the EZH2 histone methyltransferase.

In a further embodiment, the method for determining the RES phenotype of a tumor comprises determining the expression level of at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and wherein the over-expression of said genes is indicative of the RES phenotype of the tumor. Optionally, the method comprises determining the expression level of at least 20 genes selected from the group consisting of SLC16A1, SULF1, POSTN, LOX, FN1, CHI3L1, SFRP4, TNC, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C50orf13, DSC2, SFRP2, NID2, TIMP2, ADAMTS12, GPX8 and SULF2, and wherein the over-expression of said genes is indicative of the RES phenotype of the tumor. Alternatively, the method comprises determining the expression level of at least 20 genes selected from the group consisting of SLC16A1, SULF1, POSTN, LOX, FN1, CHI3L1, SFRP4, TNC, FAP, CXCL10, PLA2G7, GREM1, COL1A2, COL1A1, GUCY1A3, PFTK1, COL6A3, FBN1, IFI30, CXCL9, PRRX1, AHNAK2, and AEBP1, and wherein the over-expression of said genes is indicative of the RES phenotype of the tumor. Preferably, the method comprises determining the expression level of at least 25, 30, 35 or 40 genes selected in the above-mentioned lists. The method may comprise determining the expression level of 20, 25, 30, 35 or 40 genes selected in the above-mentioned lists. In a particular embodiment, the method comprises determining the expression level of the genes of the above-mentioned lists. In a particular aspect, the genes are selected according to the order of the list. For instance, the 20 genes may be the followings: LC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, and IFI30. The 24 genes may be the followings: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, and AEBP1. The 25 genes may be the followings: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, and GBP5. The 30 genes may be the followings: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD and CIS. The 35 genes may be the followings: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C50orf13 and DSC2. Alternatively, the genes may also be selected randomly in the list.

In addition and in the context of this embodiment, the method may further comprises determining the expression level of at least 3, 5 or 7 genes selected from the group consisting of ANXA10, IGF2, B3GALNT1, EPHB6, SEMA6A, CXorf57, SLC15A1, HS6ST3 and KRT20, and wherein the absence of over-expression of said genes is indicative of the RES phenotype of the tumor or confirms its RES phenotype. Alternatively, the method may further comprise the expression level of at least 3, 5 or 7 genes selected from the group consisting of ANXA10, IGF2, B3GALNT1, EPHB6, SEMA6A, CXorf57, SLC15A1, HS6ST3 and KRT20, and wherein the over-expression of said genes is indicative of the absence of the RES phenotype of the tumor or refutes its RES phenotype. In case discrepancy between RES+ and RES− markers, the RES status of the tumor may be determined by another method disclosed herein, preferably by the method based on the measurement of the chromosomal regions silencing. Optionally, the group may consist of the genes IGF2, B3GALNT1, EPHB6, SEMA6A, CXorf57, SLC15A1 and HS6ST3. The method may comprise determining the expression level of 3, 5, 7 or 9 genes selected in the above-mentioned lists. In a particular aspect, the genes are selected according to the order of the list. For instance, the 3 genes may be the followings: ANXA10, IGF2 and B3GALNT1. The 5 genes may be the followings: ANXA10, IGF2, B3GALNT1, EPHB6 and SEMA6A. The 7 genes may be the followings: ANXA10, IGF2, B3GALNT1, EPHB6, SEMA6A, CXorf57 and SLC15A1. Alternatively, the genes may also be selected randomly in the list.

Alternatively, the method for determining the RES phenotype of a tumor comprises determining the expression level of a first set of at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and a second set of at least 3 genes selected from the group consisting of ANXA10, IGF2, B3GALNT1, EPHB6, SEMA6A, CXorf57, SLC15A1, HS6ST3 and KRT20, and wherein the over-expression of the genes of the first set and the absence of over-expression of the genes of the second set is indicative of the RES phenotype of the tumor. Preferably, the method comprises determining the expression level of at least 25, 30, or 40 genes selected in the above-mentioned lists for the first set and of at least 5 or 7 genes selected in the above-mentioned lists for the second set. The method may comprise determining the expression level of 20, 25, 30, 35 or 40 genes selected in the above-mentioned lists for the first set and of 3, 5, 7 or 9 genes selected in the above-mentioned lists for the second set. In a particular embodiment, the method comprises determining the expression level of the genes of the above-mentioned lists. Alternatively, the genes may also be selected randomly in the list. Optionally, the method comprises determining the expression level of a first set of at least 24 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and a second set of at least 3 genes selected from the group consisting of ANXA10, IGF2, B3GALNT1, EPHB6, SEMA6A, CXorf57, SLC15A1, HS6ST3 and KRT20, and wherein the over-expression of the genes of the first set and the absence of over-expression of the genes of the second set is indicative of the RES phenotype of the tumor. More preferably, the method comprises determining the expression level of a first set of at least 24 genes consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2 and AEBP1, and a second set of at least 3 genes selected from the group consisting of ANXA10, IGF2 and B3GALNT1, and wherein the over-expression of the genes of the first set and the absence of over-expression of the genes of the second set is indicative of the RES phenotype of the tumor.

Finally, the method for determining the RES phenotype of a tumor comprises determining the expression level of at least 3, 5 or 7 genes selected from the group consisting of ANXA10, IGF2, B3GALNT1, EPHB6, SEMA6A, CXorf57, SLC15A1, HS6ST3 and KRT20, and wherein the over-expression of said genes is indicative of the absence of the RES phenotype of the tumor. Optionally, the group may consist of IGF2, B3GALNT1, EPHB6, SEMA6A, CXorf57, SLC15A1 and HS6ST3. The method may comprise determining the expression level of 3, 5, 7 or 9 genes selected in the above-mentioned lists. In a particular aspect, the genes are selected according to the order of the list. Alternatively, the genes may also be selected randomly in the list.

The expression level of a gene is determined as detailed above.

In another embodiment, the method for determining the RES phenotype of a tumor comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed, wherein the RES phenotype is defined by the presence of at least three of said over-expressed genes. Preferably, genes are selected from the group consisting of EZH2, CDC25B, TUBB3, CDH2, CXCL3, CXCL6, MLLT11, CXCL2, CTSL2, NFIL3, GPR161 and CSRP2.

The Gene ID numbers and Gene Names for the genes disclosed herein are the following:

| | | Gene |
|---|---|---|
| Gene Symbol | Gene Name | ID |
| HOXD4 | homeobox D4 | 3233 |
| HOXD3 | homeobox D3 | 3232 |
| HOXD1 | homeobox D1 | 3231 |
| MTX2 | metaxin 2 | 10651 |
| VILL | villin-like | 50853 |
| PLCD1 | phospholipase C, delta 1 | 5333 |
| DLEC1 | deleted in lung and esophageal cancer 1 | 9940 |
| ACAA1 | acetyl-CoA acyltransferase 1 | 30 |
| TCTA | T-cell leukemia translocation altered gene | 6988 |
| AMT | aminomethyltransferase | 275 |
| NICN1 | nicolin 1 | 84276 |
| DAG1 | dystroglycan 1 (dystrophin-associated glycoprotein 1) | 1605 |
| BSN | bassoon (presynaptic cytomatrix protein) | 8927 |
| APEH | N-acylaminoacyl-peptide hydrolase | 327 |
| RNF123 | ring finger protein 123 | 63891 |
| GMPPB | GDP-mannose pyrophosphorylase B | 29925 |
| SKAP2 | src kinase associated phosphoprotein 2 | 8935 |
| HOXA1 | homeobox A1 | 3198 |
| HOXA2 | homeobox A2 | 3199 |
| HOXA3 | homeobox A3 | 3200 |
| HOXA4 | homeobox A4 | 3201 |
| HOXA5 | homeobox A5 | 3202 |
| CMTM5 | CKLF-like MARVEL transmembrane domain containing 5 | 116173 |
| MYH6 | myosin, heavy chain 6, cardiac muscle, alpha | 4624 |
| MYH7 | myosin, heavy chain 7, cardiac muscle, beta | 4625 |
| THTPA | thiamine triphosphatase | 79178 |
| AP1G2 | adaptor-related protein complex 1, gamma 2 subunit | 8906 |
| DHRS2 | dehydrogenase/reductase (SDR family) member 2 | 10202 |
| DHRS4 | dehydrogenase/reductase (SDR family) member 4 | 10901 |
| CYP4F3 | cytochrome P450, family 4, subfamily F, polypeptide 3 [ | 4051 |
| CYP4F12 | cytochrome P450, family 4, subfamily F, polypeptide 12 | 66002 |
| CYP4F2 | cytochrome P450, family 4, subfamily F, polypeptide 2 | 8529 |
| CYP4F11 | cytochrome P450, family 4, subfamily F, polypeptide 11 | 57834 |
| B3GNT3 | UDP-GlcNAc:betaGal beta-1,3-N-acetylglucosaminyltransferase 3 | 10331 |
| INSL3 | insulin-like 3 (Leydig cell) | 3640 |
| JAK3 | Janus kinase 3 | 3718 |
| EZH2 | enhancer of zeste homolog 2 | 2146 |
| CDC25B | cell division cycle 25 homolog B | 994 |
| TUBB3 | tubulin, beta 3 | 10381 |
| CDH2 | cadherin 2, type 1, N-cadherin | 1000 |
| CXCL3 | chemokine (C—X—C motif) ligand 3 | 2921 |
| CXCL6 | chemokine (C—X—C motif) ligand 6 | 6372 |
| MLLT11 | myeloid/lymphoid or mixed-lineage leukemia; translocated to, 11 | 10962 |
| CXCL2 | chemokine (C—X—C motif) ligand 2 | 2920 |
| CTSL2 | cathepsin L2 | 1515 |
| NFIL3 | nuclear factor, interleukin 3 regulated | 4783 |
| GPR161 | G protein-coupled receptor 161 | 23432 |
| CSRP2 | cysteine and glycine-rich protein 2 | 1466 |
| HDAC9 | histone deacetylase 9 | 9734 |
| ANXA10 | annexin A10 | 11199 |
| SLC16A1 | solute carrier family 16, member 1 | 6566 |
| SULF1 | sulfatase 1 | 23213 |
| POSTN | periostin, osteoblast specific factor | 10631 |
| LOX | lysyl oxidase | 4015 |
| FN1 | fibronectin 1 | 2335 |
| CHI3L1 | chitinase 3-like 1 | 1116 |
| SFRP4 | secreted frizzled-related protein 4 | 6424 |
| IGF2 | insulin-like growth factor 2 | 3481 |
| TNC | tenascin C | 3371 |
| COL3A1 | collagen, type III, alpha 1 | 1281 |
| FAP | fibroblast activation protein, alpha | 2191 |
| CXCL10 | chemokine (C—X—C motif) ligand 10 | 3627 |
| PLA2G7 | phospholipase A2, group VII | 7941 |
| GREM1 | gremlin 1 | 26585 |
| COL1A2 | collagen, type I, alpha 2 | 1278 |
| COL1A1 | collagen, type I, alpha 1 | 1277 |
| GUCY1A3 | guanylate cyclase 1, soluble, alpha 3 | 2982 |
| B3GALNT1 | beta-1,3-N-acetylgalactosaminyltransferase 1 | 8706 |
| PFTK1 or | cyclin-dependent kinase 14 | 5218 |
| CDK14 | | |
| COL6A3 | collagen, type VI, alpha 3 | 1293 |
| FBN1 | fibrillin 1 | 2200 |
| IFI30 | interferon, gamma-inducible protein 30 | 10437 |
| CXCL9 | chemokine (C—X—C motif) ligand 9 | 4283 |
| PRRX1 | paired related homeobox 1 | 5396 |
| AHNAK2 | AHNAK nucleoprotein 2 | 113146 |
| AEBP1 | AE binding protein 1 | 165 |
| GBP5 | guanylate binding protein 5 | 115362 |
| MSN | moesin | 4478 |
| BGN | biglycan | 633 |
| CTHRC1 | collagen triple helix repeat containing 1 | 115908 |
| MMD | monocyte to macrophage differentiation-associated | 23531 |
| C1S | complement component 1, s subcomponent | 716 |
| IGK@ | immunoglobulin kappa locus | 50802 |
| COL5A2 | collagen, type V, alpha 2 | 1290 |
| THY1 | Thy-1 cell surface antigen | 7070 |
| C5orf13 | chromosome 5 open reading frame 13 | 9315 |
| EPHB6 | EPH receptor B6 | 2051 |
| DSC2 | desmocollin 2 | 1824 |
| SFRP2 | secreted frizzled-related protein 2 | 6423 |
| NID2 | nidogen 2 | 22795 |
| TIMP2 | TIMP metallopeptidase inhibitor 2 | 7077 |
| SEMA6A | sema domain, transmembrane domain (TM), and cytoplasmic domain, | 57556 |
| | (semaphorin) 6A | |
| CXorf57 | chromosome X open reading frame 57 | 55086 |
| SLC15A1 | solute carrier family 15 (oligopeptide transporter), member 1 | 6564 |
| HS6ST3 | heparan sulfate 6-O-sulfotransferase 3 | 266722 |
| KRT20 | keratin 20 | 54474 |
| ADAMTS12 | ADAM metallopeptidase with thrombospondin type 1 motif, 12 | 81792 |
| GPX8 | glutathione peroxidase 8 | 493869 |
| SULF2 | sulfatase 2 | 55959 |

The expression of these genes may be assessed by any method known by the skilled person such as quantitative or semi quantitative RT-PCR as well as real-time quantitative or semi quantitative RT-PCR, as described above.

In a particular embodiment, the RES phenotype is defined by the presence of at least four of said over-expressed genes.

In further embodiment, the method for determining the RES phenotype of a tumor comprises determining the number of chromosomal regions selected from the group consisting of regions 2-7, 3-2, 3-5, 7-2, 14-1, 19-3A and 19-3B which are silenced and determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed, wherein the RES phenotype is defined by the presence of at least two of said silenced regions and the presence of at least three of said over-expressed genes.

The number of silenced chromosomal regions and the number of over-expressed genes are determined as described above.

In a particular embodiment, the RES phenotype is defined by the presence of at least three of said silenced regions and the presence of at least three of said over-expressed genes.

The present invention also concerns a method for diagnosing an aggressive tumor in a subject, wherein the method comprises determining the RES phenotype in a tumor with the method according to the invention, as described above, and wherein the presence of the RES phenotype in said tumor is indicative of an aggressive tumor.

The presence of the RES phenotype in a tumor may be determined by the method of the invention as described above.

In an embodiment, the method further comprises the step of providing a sample from a subject affected with a cancer or suspected to be affected with a cancer.

In a particular embodiment, the aggressive tumor belongs to the CIS pathway.

In another embodiment, the aggressive tumor is a muscle-invasive or high grade tumor.

In a preferred embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

The present invention also concerns a method for providing useful information for the diagnosis of an aggressive tumor in a subject, wherein the method comprises determining the RES phenotype in a tumor with the method according to the invention, as described above, and wherein the presence of the RES phenotype in a tumor is indicative of an aggressive tumor. In an embodiment, the method further comprises the step of providing a sample from the subject. In a preferred embodiment, the tumor is a bladder tumor.

The inventors have herein shown that tumors with RES phenotype belong to aggressive subset of tumors. Accordingly, the present invention concerns a method for predicting or monitoring clinical outcome of a subject affected with a tumor, wherein the method comprises determining the RES phenotype in a tumor with the method according to the invention, as described above, and wherein the presence of the RES phenotype in a tumor is indicative of a poor prognosis.

In an embodiment, the method further comprises the step of providing a cancer sample from the subject.

In a particular embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

The term “poor prognosis”, as used herein, refers to an early disease progression and a decreased patient survival and/or an increased metastasis formation. This prognosis is usually associated with aggressive tumors which are frequently of high grade and progress to muscle-invasive tumors.

The inventors have herein demonstrated that tumors with the RES phenotype are particularly sensitive to epigenetic therapy. Accordingly, the present invention concerns a method for predicting the sensitivity of a tumor to an epigenetic therapy, wherein the method comprises determining the RES phenotype in said tumor with the method according to the invention, as described above, and wherein the presence of the RES phenotype in said tumor is predictive that said tumor is sensitive to an epigenetic therapy.

In an embodiment, the method further comprises the step of providing a cancer sample from the subject.

In a particular embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

In a preferred embodiment, the epigenetic therapy comprises at least one compound selected from the group consisting of histone deacetylase inhibitor, histone methyltransferase inhibitor and histone demethylase, and any combination thereof.

Preferably, the epigenetic therapy comprises at least one histone deacetylase inhibitor. More preferably, the compound is an inhibitor of histone deacetylases HDAC1, HDAC2 and/or HDAC3, more preferably of HDAC1 and/or HDAC2. Still more preferably, the epigenetic therapy comprises at least one histone deacetylase inhibitor and at least one histone methyltransferase inhibitor. In a particular embodiment, the epigenetic therapy comprises a histone deacetylase inhibitor and a histone methyltransferase inhibitor.

In a particular embodiment, the epigenetic therapy further comprises at least one DNA methyltransferase inhibitor.

A tumor is sensitive to an epigenetic therapy if the administration of such therapy induces a decreased growth rate of the tumoral cells and/or an inhibition of the growth of tumoral cells and/or the death of tumoral cells.

The present invention further concerns a method for selecting a patient affected with a tumor for an epigenetic therapy or determining whether a patient affected with a tumor is susceptible to benefit from an epigenetic therapy, wherein the method comprises determining the RES phenotype of said tumor with the method according to the invention, and wherein the presence of the RES phenotype in said tumor is predictive that an epigenetic therapy is indicated for said patient.

In an embodiment, the method further comprises the step of providing a cancer sample from the subject.

In a particular embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

In a preferred embodiment, the epigenetic therapy comprises at least one compound selected from the group consisting of histone deacetylase inhibitor, histone methyltransferase inhibitor and histone demethylase, and any combination thereof. Preferably, the epigenetic therapy comprises at least one histone deacetylase inhibitor. More preferably, the compound is an inhibitor of histone deacetylases HDAC1, HDAC2 and/or HDAC3, more preferably of HDAC1 and/or HDAC2. Still more preferably, the epigenetic therapy comprises at least one histone deacetylase inhibitor and at least one histone methyltransferase inhibitor.

In a particular embodiment, the epigenetic therapy further comprises at least one DNA methyltransferase inhibitor.

The present invention also concerns an epigenetic compound for use in the treatment of cancer in a patient affected with a tumor with a RES phenotype.

The presence of the RES phenotype in a tumor may be assessed by any method of the invention, as described above.

In an embodiment, the epigenetic compound is selected from the group consisting of histone deacetylase inhibitor, histone methyltransferase inhibitor and histone demethylase, and any combination thereof.

In a preferred embodiment, the epigenetic compound is a histone deacetylase inhibitor. Preferably, the compound is an inhibitor of histone deacetylases HDAC1, HDAC2 and/or HDAC3, more preferably of HDAC1 and/or HDAC2. More preferably, the histone deacetylase inhibitor is used in combination with a histone methyltransferase inhibitor.

In a particular embodiment, the epigenetic compound is used in combination with a DNA methyltransferase inhibitor.

In another particular embodiment, the epigenetic compound is used in combination with an antineoplastic agent.

An “antineoplastic agent” is an agent with anti-cancer activity that inhibits or halts the growth of cancerous cells or immature pre-cancerous cells, kills cancerous cells or immature pre-cancerous cells, increases the susceptibility of cancerous or pre-cancerous cells to other antineoplastic agents, and/or inhibits metastasis of cancerous cells. These agents may include chemical agents as well as biological agents. Examples include, without limitation, 5-aza-2′deoxycytidine, 17-AAG (17-N-Allylamino-17-demethoxygeldanamycin), tretinoin (ATRA), bortezomib, cisplatin, carboplatin, oxaliplatin, paclitaxel, bevacizumab, tamoxifen, leucovorin, docetaxel, transtuzumab, etoposide, flavopiridol, 5-fluorouracil, irinotecan, TRAIL (TNF-related apoptosis-inducing ligand), LY294002, PD184352, perifosine, Bay 11-7082, gemcitabine, bicalutamide, zoledronic acid, cis-retinoic acid, MK-0457, imatinib, desatinib, sorafenib, temozolomide, actinomycin, anthracyclines, doxorubicin, daunorubicin, valrubicine, idarubicine, epirubicin, bleomycin, plicamycin and mitomycin. Antineoplastic agents may also include radiotherapeutic agents such as X-rays, gamma rays, alpha particles, beta particles, photons, electrons, neutrons, radioisotopes, and other forms of ionizing radiation.

In a particular embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor.

The present invention further concerns a method for treating a cancer in a patient affected with a tumor with a RES phenotype, said method comprising the administration of a therapeutically effective amount of an epigenetic compound to said patient.

The term “therapeutically effective amount” refers to that amount of a therapy which is sufficient to reduce or ameliorate the severity, duration and/or progression of a disease or one or more symptoms thereof. As used herein, this term refers to that amount of an epigenetic compound which is sufficient to destroy, modify, control or remove primary, regional or metastatic cancer tissue, ameliorate cancer or one or more symptoms thereof, or prevent the advancement of cancer, cause regression of cancer, or enhance or improve the therapeutic effect (s) of another therapy (e.g., a therapeutic agent). This term may also refer to the amount of an epigenetic compound sufficient to delay or minimize the spread of cancer or sufficient to provide a therapeutic benefit in the treatment or management of cancer. Further, a therapeutically effective amount with respect to an epigenetic compound means that amount of epigenetic compound alone, or in combination with other therapeutic agent, that provides a therapeutic benefit in the treatment or management of cancer.

In an embodiment, the method further comprises determining the RES phenotype of said tumor with the method of the present invention as described above.

In a particular embodiment, the tumor is selected from the group consisting of bladder cancer, colorectal cancer, oesophageal cancer, neuroblastoma, breast cancer and lung cancer. Preferably, the tumor is selected from the group consisting of bladder cancer, colorectal cancer and breast cancer. More preferably, the tumor is a bladder tumor. In an embodiment, the epigenetic compound is selected from the group consisting of histone deacetylase inhibitor, histone methyltransferase inhibitor and histone demethylase, and any combination thereof. Preferably, the epigenetic compound is a histone deacetylase inhibitor. More preferably, the compound is an inhibitor of histone deacetylases HDAC1, HDAC2 and/or HDAC3, more preferably of HDAC1 and/or HDAC2. Still more preferably, the histone deacetylase inhibitor is administrated simultaneously or sequentially with a histone methyltransferase inhibitor.

The present invention also concerns:
a kit for determining the RES phenotype of a tumor, wherein the kit comprises detection means selected from the group consisting of a pair of primers, a probe and an antibody specific to a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2; or to b) the genes EZH2, CDC25B, TUBB3, CDH2, CXCL3, CXCL6, MLLT11, CXCL2, CTSL2, NFIL3, GPR161, CSRP2 and HDAC9; or
a DNA chip for determining the RES phenotype of a tumor, wherein the DNA chip comprises a solid support which carries nucleic acids that are specific to a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2; or to b) the genes EZH2, CDC25B, TUBB3, CDH2, CXCL3, CXCL6, MLLT11, CXCL2, CTSL2, NFIL3, GPR161, CSRP2 and HDAC9.

In a particular embodiment, the kit or DNA chip comprises detection means or nucleic acids that are specific to:
a) at least 20 genes selected from the group consisting of SLC16A1, SULF1, POSTN, LOX, FN1, CHI3L1, SFRP4, TNC, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8 and SULF2; or
b) at least 20 genes selected from the group consisting of SLC16A1, SULF1, POSTN, LOX, FN1, CHI3L1, SFRP4, TNC, FAP, CXCL10, PLA2G7, GREM1, COL1A2, COL1A1, GUCY1A3, PFTK1, COL6A3, FBN1, IFI30, CXCL9, PRRX1, AHNAK2, and AEBP1; or
c) the following 20 genes: LC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, and IFI30;
d) the following 24 genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, and AEBP1; or
e) the following 25 genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, and GBP5; or
f) the following 30 genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD and CIS; or
g) the following 35 genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13 and DSC2; or
h) the following genes: SLC16A1, SULF1, POSTN, LOX, FN1, CHI3L1, SFRP4, TNC, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8 and SULF2.

Optionally, the kit or DNA chip may further comprise detection means or nucleic acids that are specific to at least 3, 5 or 7 genes selected from the group consisting of ANXA10, IGF2, B3GALNT1, EPHB6, SEMA6A, CXorf57, SLC15A1, HS6ST3 and KRT20. In particular, the kit or DNA chip may further comprise detection means or nucleic acids that are specific to ANXA10, IGF2 and B3GALNT1.

Accordingly, the present invention relates to the kit or DNA chip comprising detection means or nucleic acids that are specific to:
a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and a second set of at least 3 genes selected from the group consisting of ANXA10, IGF2, B3GALNT1, EPHB6, SEMA6A, CXorf57, SLC15A1, HS6ST3 and KRT20; or
b) at least 24 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, and a second set of at least 3 genes selected from the group consisting of ANXA10, IGF2, B3GALNT1, EPHB6, SEMA6A, CXorf57, SLC15A1, HS6ST3 and KRT20; or
c) the following genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, ANXA10, IGF2 and B3GALNT1; or
d) the following genes: SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2, NID2, TIMP2, ADAMTS12, GPX8, SULF2, ANXA10, IGF2, B3GALNT1, EPHB6, SEMA6A, CXorf57, SLC15A1, HS6ST3 and KRT20.

Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labeled and contacted with the microarray in hybridization conditions, leading to the formation of complexes between target nucleic acids that are complementary to probe sequences attached to the microarray surface. The labeled hybridized complexes are then detected and can be quantified or semi-quantified. Labeling may be achieved by various methods, e.g. by using radioactive or fluorescent labeling. Many variants of the microarray hybridization technology are available to the man skilled in the art (see e.g. the review by Hoheisel, et 2006).

The kit or DNA chip of the invention includes detection means for the genes as defined above in the method for determining the RES phenotype. In a particular aspect, the kit or DNA chip does not include means for detecting more than 100, 80, 70, or 60 genes.

The kit or DNA chip of the invention can further comprise detection means or nucleic acids for control gene, for instance a positive and negative control or a nucleic acid for an ubiquitous gene in order to normalize the results.

All references cited in this specification are incorporated by reference.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.”

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

The following examples are given for purposes of illustration and not by way of limitation.

### EXAMPLES

### Example 1

### Materials and Methods

### Patients and Tissue Samples

The analysis of the gene expression profiles and genomic alterations of 57 urothelial bladder carcinomas have been previously reporter (Stransky et al., 2006). These carcinomas were obtained from 53 patients included between 1988 and 2001 in the prospective database established in 1988 at the Department of Urology of Henri Mondor Hospital. The tumor samples came from 16 Ta, 9 T1, 6 T2, 13 T3 and 13 T4 tumors. The flash-frozen tumor samples were stored at −80° C. immediately after transurethral resection or cystectomy. All tumor samples contained more than 80% tumor cells, as assessed by H&E staining of histological sections adjacent to the samples used for transcriptome and genome analyses. Five normal urothelial samples, obtained as described in the article of Diez de Medina et al. (Diez de Medina et al., 1997) were also used for transcriptome analysis. An independent set of 40 human bladder tumors, containing 10 Ta, 6 T1, 6 T2, 7 T3 and 11 T4 tumors, was used to validate the existence of the RES phenotype. These tumors, provided by the Henri Mondor and Foch hospitals and Institut Gustave Roussy, were obtained from 40 patients who underwent surgery between 1993 and 2006. All patients provided informed consent and the study was approved by the ethics committees of the different hospitals.

### RNA and DNA Extraction

RNA and DNA were extracted from the samples by cesium chloride density centrifugation (Chirgwin et al., 1979). The concentration and integrity/purity of each RNA sample were determined with the RNA 6000 LabChip Kit (Agilent Technologies) and an Agilent 2100 bioanalyzer. DNA purity was also assessed from the ratio of absorbances at 260 and 280 nm. DNA concentration was determined with a Hoechst dye-based fluorescence assay49. RNA and DNA were extracted from cell lines with Qiagen extraction kits (Qiagen, Courtaboeuf, France).

### Cell Culture

The bladder cancer cell lines TCCSUP, HT1376, RT112, T24, MGHU3 and CL1207 were cultured in DMEM F-12 Glutamax medium supplemented with 10% FCS; JMSU1 cells were cultured in RPMI Glutamax medium supplemented with 10% FCS. Normal human urothelial (NHU) cells were established as finite cell lines and cultured in complete keratinocyte serum-free medium, as described in the article of Southgate et al (Southgate et al., 1994). In these experiments, two independent NHU cell lines were used at passage 4. For analyses of the effect of trichostatin A (TSA) (Calbiochem, Fontenay-sous-Bois, France) and/or 5-aza-deoxycytidine (Calbiochem) on transcript expression, normal and tumor cells were seeded in 25 cm² dishes at a density of 8×10⁵ cells/dish. Cultures were treated the next day with 300 nM trichostatin A (TSA) for 16 hours, 5 μM 5-aza-deoxycytidine for 72 hours, or 5 μM 5-aza-deoxycytidine for 48 hours followed by 300 nM TSA for 10 hours. These experiments were repeated twice and each time, each condition was tested in duplicate.

### Trichostatin A Sensitivity Assays

All bladder cancer and NHU cell lines were seeded in 12-well plates at a density of 5×10⁴ cells/well. Cultures were treated the next day, in duplicate, with various doses of trichostatin A, from 100 nM to 500 nM, with two wells left untreated. After 72 hours, the living cells in each treated well were harvested and counted and compared to the numbers of cells in the non-treated wells. The resulting ratio was used to assess sensitivity to trichostatin A.

### FGFR3 Mutation Analysis

FGFR3 mutations were studied using the SNaPshot technique as described in Van Oers et al. (Van Oers et al., 2005).

### Quantitative RT-PCR

1 μg of total RNA was used for reverse transcription, with random hexamers (20 pmol) and 200 U MMLV reverse transcriptase. To assess mRNA levels by real-time quantitative PCR (RT-qPCR), we used either individual assays or the TaqMan Low Density Array (TLDA) on an ABI PRISM 7900 real-time thermal cycler (Applied Biosystems, Foster City). With both methods, all samples were run in duplicate. For all experiments involving T1207 and CL1207 both methods were used. For individual assays, the SYBR Green kit was used to measure the expression of the RNAs of interest and the Taqman kit (Applied Biosystems) for the reference RNAs (18S rRNA). For TLDA, the same reference 18S was used; predesigned TaqMan probe and primer sets for the different genes were chosen from the Applied Biosystems catalogue. Amounts of mRNAs of the genes of interest were normalized to that of the reference gene according to the 2^{-ΔCt} method.

### DNA Methylation Analysis

The methylation status of the promoters was assessed by bisulfite sequencing and COBRA (Xiong et al., 1997). Briefly, 2 μg of genomic DNA was treated with sodium bisulfite, purified using the Epitect kit (Qiagen) and amplified as follows: initial incubation at 94° C. for 4 minutes, followed by 35 cycles of denaturation at 94° C. for 30 seconds, annealing at Tm for 30 seconds and extension at 72° C. for 30 seconds, using Biolabs Taq Polymerase (Ozyme, Saint-Quentin-en-Yvelines, France). For bisulfite sequencing, the purified PCR product was cloned using TA cloning kit (Invitrogen, Cergy Pontoise, France) and ten clones for each sample and gene were sequenced. For COBRA, the PCR products were digested for 16 hours with a restriction enzyme recognizing a restriction site containing a CpG dinucleotide. The corresponding CpG site is inferred as methylated when the PCR product is digested.

### Chromatin Immunoprecipitation

Chromatin immunoprecipitation (ChIP) assays were carried out in duplicate in three 150 cm² dishes for untreated CL1207, CL1207 treated with 300 nM TSA for 16 h, TCCSUP, RT112 and NHU cells. Chromatin was prepared with an enzymatic kit (Active Motif, Rixensart, Belgium). An extract of the original chromatin was kept as an internal standard (Input DNA). The complexes were immunoprecipitated with 4 μg of antibodies against trimethyl histone H3 (Lys27) (Upstate Biotechnology, Santa Cruz), trimethyl histone H3 (Lys9) (Abcam, Cambridge, UK) or acetyl histone H3 (Lys9) (Abcam). The amount of immunoprecipitated target was determined by real-time PCR, in duplicate, using the ABI PRISM 7900HT Sequence Detection System. For each sample and each promoter, an average C_{T} value was obtained for immunoprecipitated material and for the input chromatin. The amount of immunoprecipitated material was defined as 2̂(C_{T}(Input DNA)−C_{T}(Immunoprecipitated DNA)).

### Affymetrix Array Analyses

For all Affymetrix array expression analyses, Affymetrix MASS signal values were Log 2-transformed and normalized by removing chip-specific and probe set-specific effects (the mean signal for all probe sets across one chip and the mean signal for one probe set across all chips, respectively). Statistical analysis and numerical calculations were carried out with R 2.6 (R Foundation for Statistical Computing) and Amadea® (Isoft, Gif-sur-Yvette, France).

### Clustering Analyses

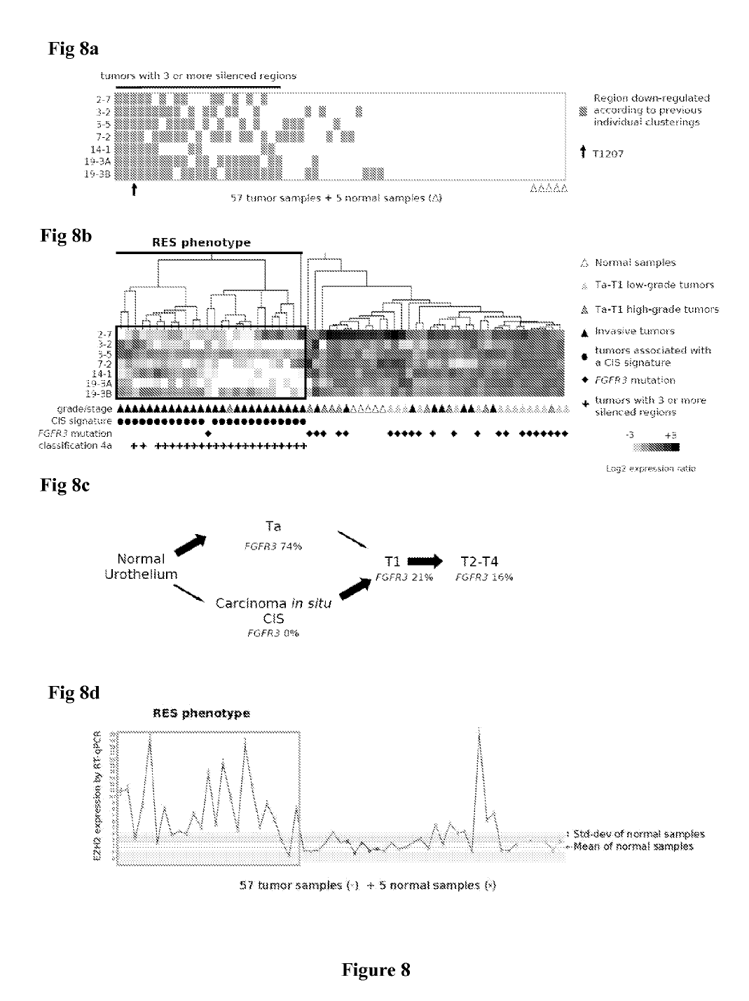

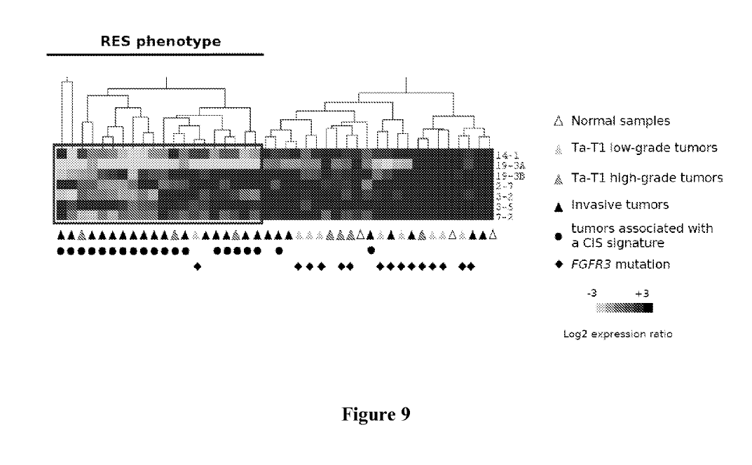

Cluster analyses were used (i) to identify, from Affymetrix expression data, regions of correlated expression, independent of copy number changes, which presented an up or downregulation in subsets of tumor samples, (ii) to identify tumors with the RES phenotype using Affymetrix (FIG. 8b) or RT-qPCR expression data (FIG. 9) and (iii) to identify tumors with the CIS signature using Affymetrix or RT-qPCR expression data. For (ii) and (iii), the Affymetrix data were used for the test group (n=57 tumors +5 normal samples) and the RT-qPCR data for the validation group (n=40 tumors +3 normal samples). The Cluster 3.0 program (Eisen et al., 1998) was used for hierarchical clustering. Results were displayed using the TreeView program (Eisen et al., 1998).

### Defining the RES Phenotype

Two different methods were used to define the RES phenotype.

(1) Using individual clustering (FIG. 8a): a region was considered as down-regulated in a given sample if, in the individual clustering for this region, the sample belonged to the cluster arm of downregulated tumors.

(2) Using the region expression score (FIG. 8b): for each sample and each region, expression levels of all the genes in the region were Log 2-transformed and normalized; the region expression score was calculated as the average difference between this sample and normal values. Tumors were clustered according to these region expression scores.

### Results

### Identification of Downregulated Chromosomal Regions in Bladder Tumors

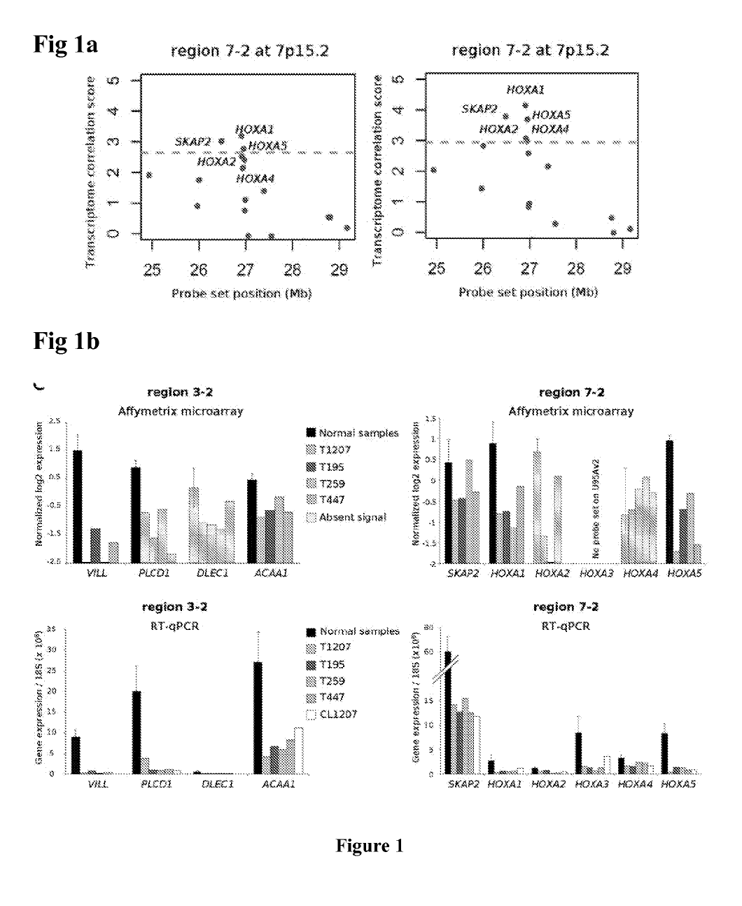

By combining transcriptome and CGH array data for a set of 57 bladder carcinomas of varying grade and stage, 28 copy number-independent regions of correlated expression have been previously identified (Stransky et al., 2006). The strategy used is summarized by the example in FIG. 1a. In the left panel, the transcriptome correlation map (TCM) of a part of chromosome 7 at 7p15.2 is shown. This map assesses the correlation which exists between the expression of a gene and those of its neighbors (Reyal et al., 2005). Based on this correlation map, region 7-2 at 7p15.2, which displayed correlated expression was identified: the genes indicated above the dashed line in this figure have a high transcriptome correlation score indicating that within this region, expression of each gene is significantly correlated to that of its neighbors (p<0.002) (Reyal et al., 2005). CGH array analyses of the same tumor set led to the identification of tumors that showed genetic losses or gains in this region (data not shown). From this was calculated a new TCM that excluded tumors with copy-number alterations (FIG. 1a, right panel). The three genes of region 7-2 present on the initial map (SKAP2, HOXA1 and HOXA5) remained correlated in the recalculated map, indicating that the correlation within this region was copy number-independent. Two additional correlated genes (HOXA2 and HOXA4) were identified in this second map, just above the threshold after TCM recalculation.

The inventors next investigated whether within each of the 28 regions, the correlated expression of genes was due to down and/or upregulation, and whether for each region, the deregulation was represented by all or a subset of tumors. Thus, for each region a clustering analysis of tumor and normal samples was performed according to the expression of the correlated genes, as determined by Affymetrix arrays. For each correlated gene, the ratio between its expression value in each sample and its mean expression in normal urothelium was calculated (n=5). These expression ratios were then used to cluster, for each region, all normal and tumor samples. To look for regions of downregulation (or upregulation), tumor samples with genetic losses (or gains) in these regions were excluded from the clustering analysis. This analysis identified several categories of region. For some regions, the correlated expression of genes was due to a downregulation, with this downregulation affecting only a subset of tumors. Other regions were upregulated in a subset of tumors. A third group of regions was downregulated in some tumors and upregulated in others. The remaining regions displayed no clear expression pattern. Of the 28 copy number-independent regions of correlation, seven displayed only downregulation (regions 1-1, 3-2, 3-5, 6-7, 7-2, 14-1 and 19-3). Region 19-3 could be sub-divided into two sub-regions of downregulation (19-3A and 19-3B), as cluster analysis showed that these two sub-regions were separated by 1.3 Mb which contained several genes that displayed normal expression values. Two regions (regions 2-7 and 17-7) were subjected to both down- and upregulation and six were subjected only to upregulation (1-6, 2-3, 4-2, 5-3, 6-3 and 12-4). As the inventors were interested in regions that were possibly subject to epigenetic silencing, they focused subsequent analysis on the 10 regions which presented downregulation (regions 1-1, 2-7, 3-2, 3-5, 6-7, 7-2, 14-1, 17-7, 19-3A and 19-3B).

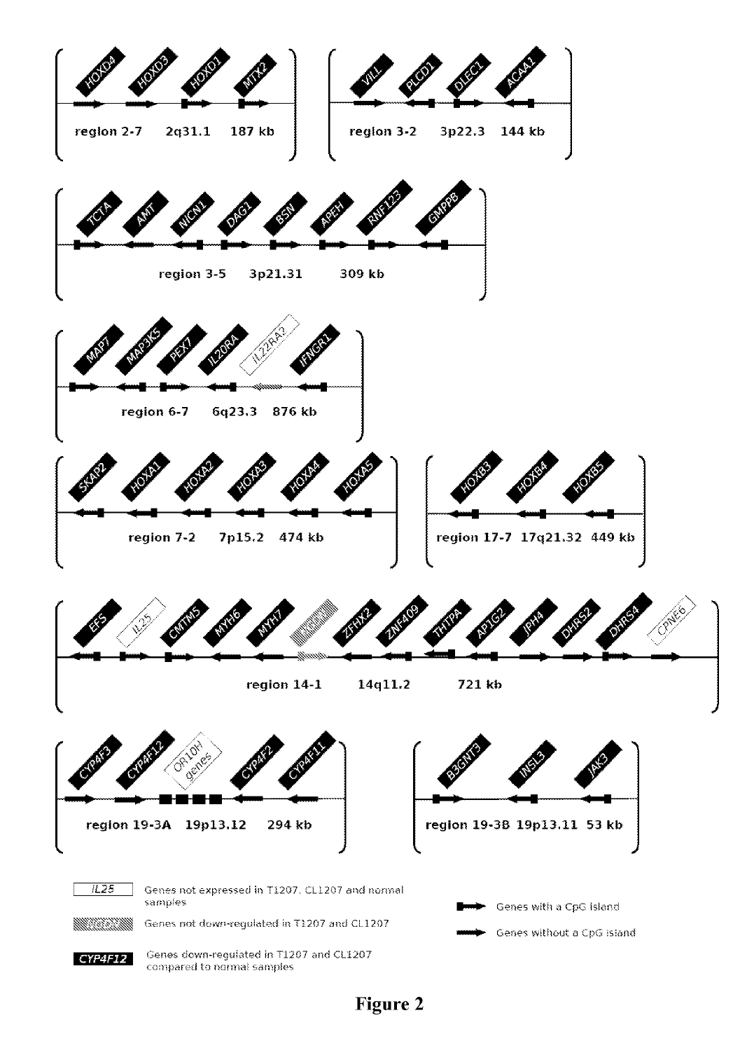

To determine if the downregulation in these 10 regions affected stretches of contiguous genes, an extensive study of the expression of all genes within these regions was performed by RT-qPCR, analyzing both the genes present and not present on Affymetrix U95A arrays. This analysis was carried out on tumor T1207 and a cell line derived from this tumor, CL120716. Tumor T1207 was chosen because it showed downregulation in all 10 regions as shown by Affymetrix data (data not shown), and it did not present any genetic loss in these regions, as shown by CGH array (data not shown). Also, the availability of a cell line from this tumor allowed subsequent functional analyses. FIG. 1b indicates the Affymetrix and RT-qPCR data for regions 3-2 and 7-2 for tumor T1207, the cell line CL1207 and for samples of normal urothelium. Three additional tumors (T195, T259, T447), which were identified as showing transcript downregulation without genetic loss for these two regions, were also analyzed (FIG. 1b). The genes comprising region 3-2 (VILL, PLCD1, DLEC1, ACAA1) were all represented on the Affymetrix array. RT-qPCR analysis confirmed that the genes were downregulated in all four tumors and in the cell line CL1207; this included DLEC1, which was scored as absent by the Affymetrix software MAS5 (FIG. 1b). In the case of region 7-2, which contains the genes SKAP2, HOXA1, HOXA2, HOXA3, HOXA4, RT-qPCR indicated that all the genes were downregulated in all tumor samples. The Affymetrix data were in good agreement with the RT-qPCR data for the genes SKAP2, HOXA1 and HOXA5 which were scored by MAS5 as present in normal urothelium. The other genes were either tagged by MAS5 as absent (HOXA2, HOXA4), or had no probe set on the Affymetrix chip (HOXA3). The RT-qPCR data for the genes within the 10 regions of downregulation from tumor T1207 and its derived cell line CL1207 are not shown. Three or more contiguous genes showing downregulation were considered to be a “stretch” of downregulated genes. Genes not expressed in normal urothelium and in the tumor were included in the stretches. Overall, for all of the 10 regions analyzed, other than region 1-1, stretches of contiguous downregulated or non-expressed genes were observed. All stretches are presented in FIG. 2. These stretches varied in length from 53 kb to 876 kb. In region 14-1, a single gene with unaltered expression, NGDN, was located within a stretch of 10 genes that were downregulated or not expressed.

Re-Expression of the Downregulated Regions Following Treatment with 5-Azadeoxycytidine and/or TSA

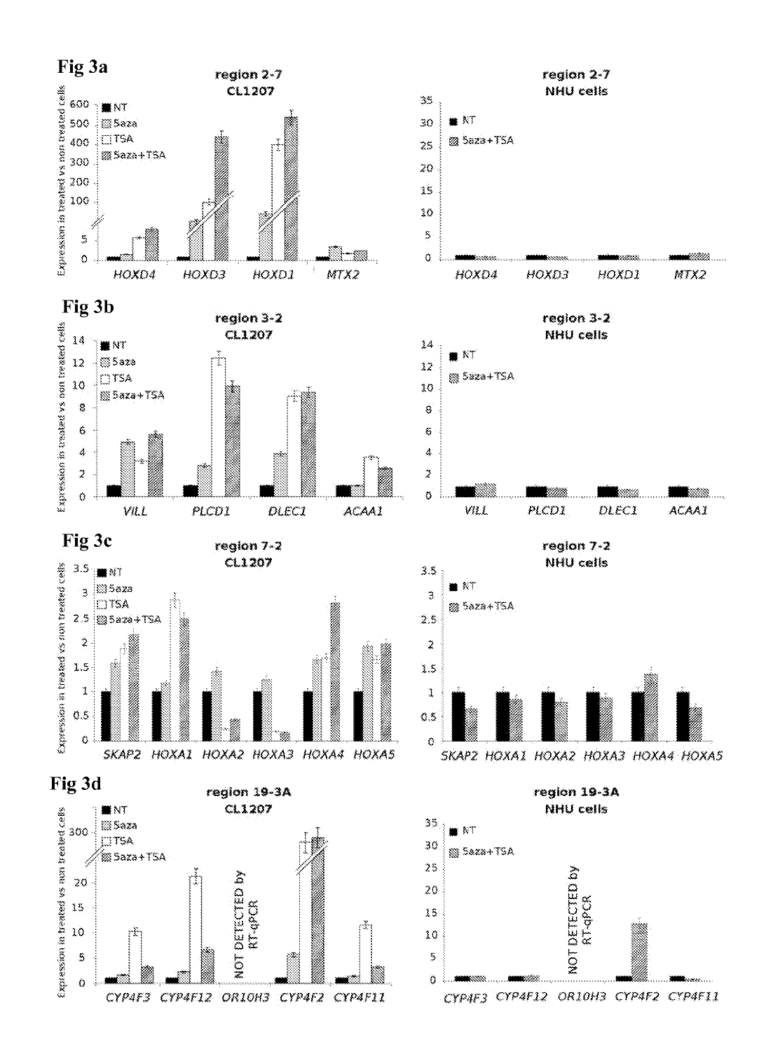

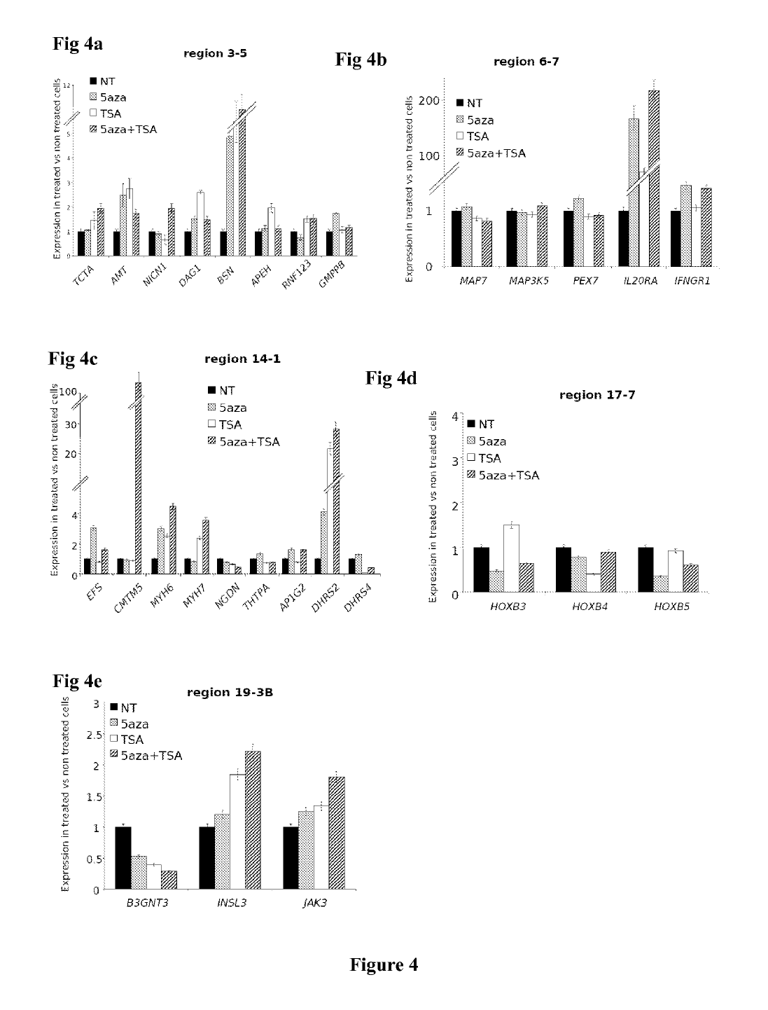

Tumor T1207 and its derived cell line CL1207 presented identical downregulation profiles. CL1207 was therefore used to investigate whether all genes within the nine silenced stretches were coordinately affected by an epigenetic mechanism. In particular, it was tested whether DNA methylation and/or histone acetylation/methylation might be involved. Firstly, CL1207 cells were treated with the DNA demethylating agent, 5-aza-deoxycytidine, and/or with the histone deacetylase inhibitor, trichostatin A (TSA). These different treatments led to reexpression of most of the genes in seven regions (2-7, 3-2, 3-5, 7-2, 14-1, 19-3A and 19-3B) (FIG. 3a-d and FIG. 4a-e). The results for regions 2-7, 3-2, 7-2 and 19-3A are shown in FIG. 3 (left panels). All genes in regions 2-7, 3-2 and 19-3A were re-expressed (FIG. 3a, b and d). Four of the six genes in region 7-2 were re-expressed after treatment (FIG. 3c). The effect of 5-aza-deoxycytidine plus TSA treatment was also studied in normal human urothelial cells (NHU cells) grown in culture (Southgate et al., 1994) (FIG. 3 right panels and data not shown). No re-expression was observed, except for some isolated genes, for example CYP4F2 in region 19-3A (FIG. 3d, right panel).

In two regions (regions 6-7 and 17-7), treatment of CL1207 cells with 5-azadeoxycytidine and/or TSA led either to no re-expression or re-expression of only one isolated gene (FIGS. 4b and d); this suggests that these regions were not silenced by DNA methylation or histone hypoacetylation/methylation. These two regions were therefore excluded from subsequent analyses.

The Silencing of Entire Chromosomal Regions is Associated with Abnormal Histone Modification Patterns

The possible involvement of DNA methylation and/or histone hypoacetylation/methylation in the silencing of the seven regions re-expressed after treatment with 5-aza-deoxycytidine and/or TSA was investigated.

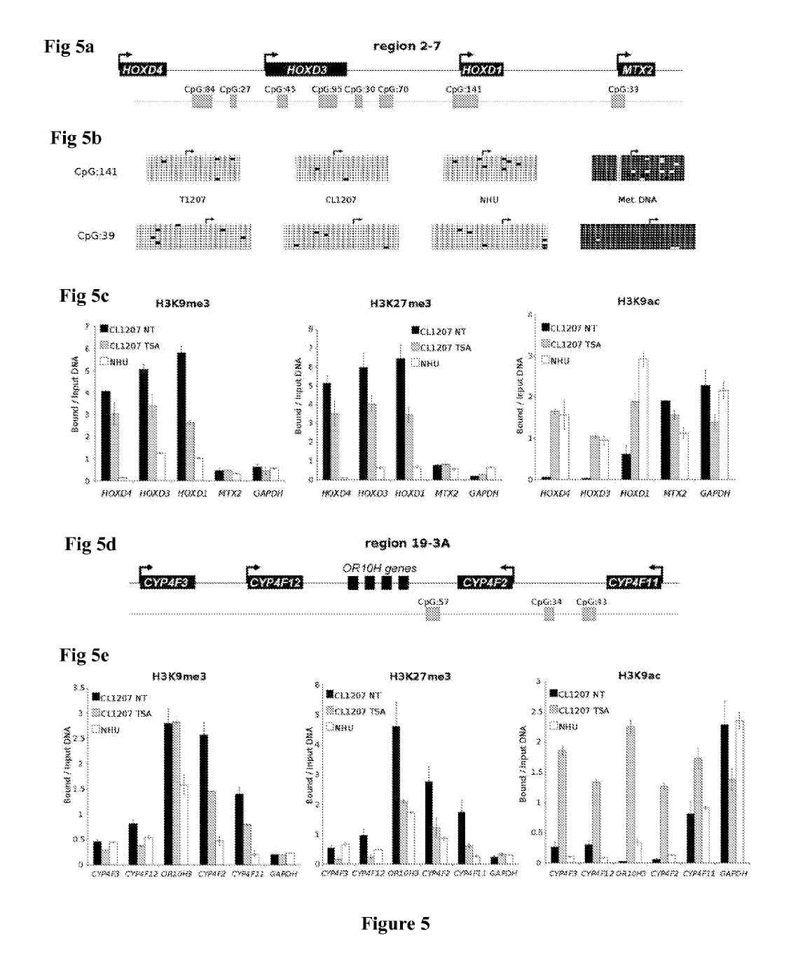

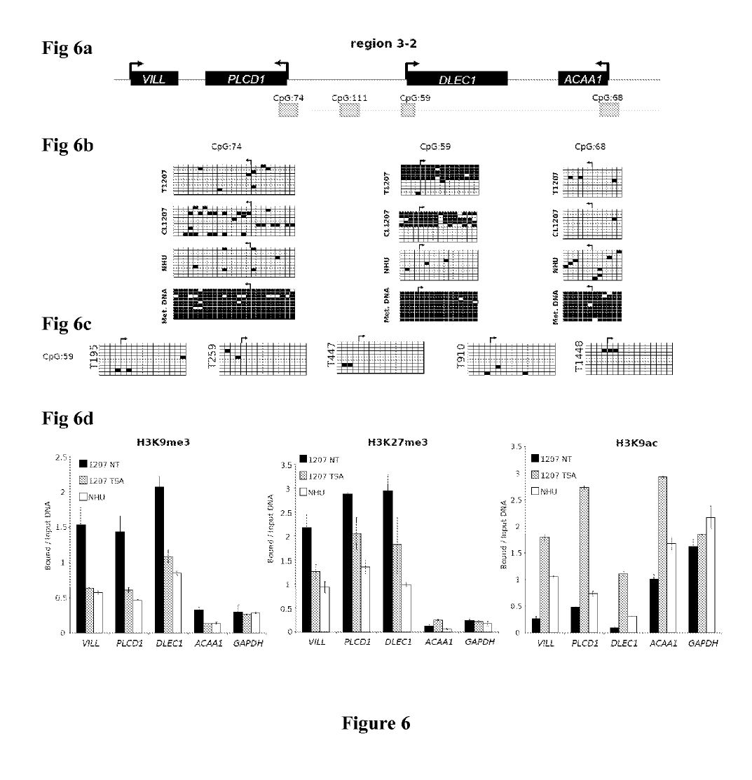

DNA methylation and histone modifications (H3K9me3, H3K27me3 and H3K9ac) were analyzed in detail for three of these regions (regions 2-7, 3-2 and 19-3A) (FIG. 5 and FIG. 6).

The DNA methylation status of CpG islands associated with promoters was examined in tumor T1207 and its derived cell line CL1207 by bisulfite sequencing. DNA from NHU cells and fully-methylated DNA were used for comparison. The results are shown for region 2-7 (FIGS. 5a and b) and for region 3-2 (Supplementary FIGS. 6a and b). Region 19-3A did not contain any gene with a promoter-associated CpG island. For region 2-7, the promoter associated CpG islands (CpG 141 around the HOXD1 promoter and CpG 39 around the MTX2 promoter) were not methylated in T1207, CL1207 or NHU cells. Three genes in region 3-2 had a promoter-associated CpG island (PLCD1, DLEC1 and ACAA1; FIG. 6a): the PLCD1 and ACAA1 promoters were not significantly methylated; the DLEC1 promoter was hemi-methylated in T1207 and CL1207 (FIG. 6b middle panel). To understand whether methylation was necessary to the downregulation of DLEC1, the methylation of the DLEC1 promoter was studied in five more tumors displaying a downregulation of region 3-2 (including T195, T259, and T447 shown in FIG. 1b left panel) and found no methylation (FIG. 6c)

Histone modifications in the cell line CL1207 in the promoter regions of the genes located in these three regions (regions 2-7, 19-3A, 3-2) were then investigated, using chromatin immunoprecipitation (ChIP) followed by qPCR. Antibodies specific for two inactive marks (trimethylation of Lys9 of histone H3 (H3K9me3) and trimethylation of Lys27 of histone H3 (H3K27me3)) and for one active mark (acetylation of Lys9 of histone H3, H3K9ac) were used (FIGS. 5c and e and FIG. 6d). The histone modifications assessed in CL1207 were measured before and after treatment with the histone deacetylase inhibitor TSA. The histone modifications were also assessed for comparison on the promoters of the same genes in NHU cells grown in culture and in the promoter of an ubiquitously expressed gene (GAPDH). Most promoters of the genes in the three regions displayed high levels of the two repressive marks (H3K9me3 and H3K27me3) in CL1207 cells in comparison to the ubiquitously expressed GAPDH gene and in comparison to normal NHU cells (FIGS. 5c and e and FIG. 6d). The promoters of the genes in regions 2-7, 3-2 and 19-3A were hypoacetylated at H3K9 in CL1207 cells relative to the promoter of the GAPDH gene. Acetylation levels for regions 2-7 and 3-2, but not region 19-3A, were higher in NHU cells than CL1207 cells. TSA treatment of CL1207 decreased the levels of the inactive marks and increased the levels of the active mark for most of the genes in all three regions. These changes correlated with the increase in the expression of the genes in these three regions following TSA treatment (FIG. 3).

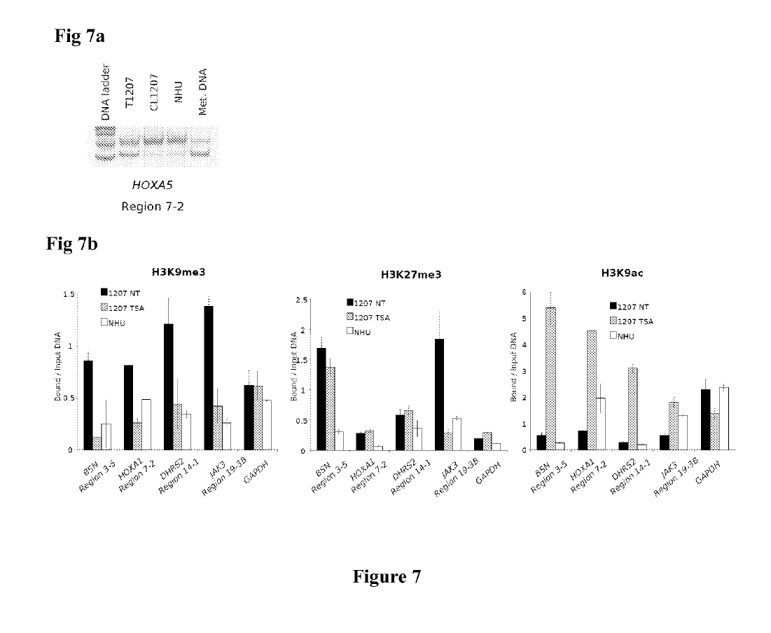

DNA methylation and the same histone modifications (H3K9me3, H3K27me3 and H3K9ac) were also analyzed for the four other silenced regions (3-5, 7-2, 14-1 and 19-3B). In this case, the COBRA method (Xiong et al., 1997) was used and the DNA methylation studies were restricted to the CpG islands around promoters of the genes re-expressed after 5-aza-deoxycytidine treatment alone (FIG. 3 and FIG. 4), as this indicated genes possibly controlled by DNA methylation: BSN in region 3-5, SKAP2, HOXA4 and HOXA5 in region 7-2, EFS and AP1G2 in region 14-1. DNA methylation was observed only for HOXA5 (region 7-2; FIG. 7a and data not shown). However, the promoter of HOXA5 was methylated in T1207, but not in CL1207. These results showed that promoter DNA methylation was not an essential part of the silencing process in this case. The inventors looked in the four regions for the same histone modifications (H3K9me3, H3K27me3, H3K9ac), limiting their analysis to one gene in each region (FIG. 7b) and comparing non-treated CL1207 cells to TSA-treated CL1207 and NHU cells. It was found that CL1207 cells showed high levels of H3K9 trimethylation in the promoters of BSN (region 3-5), HOXA1 (region 7-2), DHRS2 (region 14-1) and JAK3 (region 19-3B), as well as H3K27 trimethylation in promoters of BSN and JAK3; these marks were decreased after treatment by TSA. All four promoters also lacked acetylation on lysine 9 in CL1207 cells.

These results showed that the seven identified regions of downregulation were silenced by an epigenetic mechanism involving histone modifications. Promoter DNA methylation was very rare and when present was not significant enough to explain the silencing of these regions.

Identification of a Regional Epigenetic Silencing Phenotype Associated with Muscle-Invasive Bladder Carcinomas

The inventors have shown that the same tumor T1207 showed simultaneous epigenetic downregulation of all seven regions (2-7, 3-2, 3-5, 7-2, 14-1, 19-3A and 19-3B). In addition, cluster analysis had indicated that for each of the seven regions, downregulation was restricted to specific subsets of tumors. To determine if common silencing of the different regions occurred in the same group of bladder tumors, it was first tested whether these subsets of tumors overlapped. In FIG. 8a, for each of the seven regions of epigenetic silencing, it was indicated which of the 57 tumors displayed downregulation. Thirty-four tumors had two or fewer silenced regions, whereas 23 tumors had three or more silenced regions, suggesting the existence of a regional epigenetic silencing (RES) phenotype. A second approach was used to define more precisely the two groups of tumors: those with and without the RES phenotype (FIG. 8b). Firstly, for each tumor sample in a given region, a region expression score was calculated: this score evaluated, for each sample and each region, the mean fold-change in expression compared to normal urothelium (see Methods). A cluster analysis was then carried out: tumors and normal samples were clustered according to their region expression scores (FIG. 8b). Twenty-six tumors (including the 23 previously identified in FIG. 8a) clustered together and presented downregulation of all or several of the seven regions. This group of tumors defined the RES phenotype. Significantly, 25 of these 26 samples (96%) were muscle-invasive (≧T2), with the remaining sample corresponding to a high-grade (G3) T1 tumor. The group of samples that did not display the RES phenotype (31 tumors and 5 normal urothelial samples) included the seven remaining muscle-invasive tumors, all but one of the high-grade Ta and Ti tumors (7 of 8), all low-grade (G1 and G2) Ta and T1 tumors (n=16), and all normal samples (n=5).

Most muscle-invasive tumors (T2-4) develop from carcinoma in situ (CIS) (Wu et al. 2005) as illustrated in FIG. 8c. To assess which tumors were derived from CIS in our series of 57 bladder tumors, it was analyzed which tumors were associated with the CIS signature previously defined (Dyrsjkot et al., 2004). This signature was determined using the 61 genes present on the Affymetrix U95A array out of the 100 genes previously defined (Dyrsjkot et al., 2004) (Data not shown). Twenty-five of the 57 tumors presented the CIS signature, and remarkably, all 25 displayed the RES phenotype. Only one tumor displayed the RES phenotype, but not the CIS signature. The second pathway of bladder cancer progression involves development of Ta tumors, usually of low grade, which progress rarely to muscle-invasive tumors (FIG. 8c). This pathway is associated with a high frequency of activating FGFR3 mutations, whereas CIS-associated tumors have few if any such mutations (Billerey et al., 2001). In our series of 57 tumors, 23 tumors had an FGFR3 mutation, and all but one of these tumors belonged to the group lacking the RES phenotype.

Six of the seven regions defining the RES phenotype presented H3K27 trimethylation, the footprint of the EZH2 methyl-transferase. EZH2 mRNA levels in the 57 tumors (as determined by RT-qPCR analyses) were then compared with those in normal urothelia. Nineteen of the 26 tumors with RES phenotype, but only five tumors without the RES phenotype presented a significant over-expression of EZH2 (FIG. 8d).

The existence of the RES phenotype and its association with aggressive bladder tumors of the CIS pathway was validated in an independent set of 40 bladder tumors of various stages and grades. The expression of all genes within the seven identified regions along with the genes that define the CIS signature (Dyrsjkot et al., 2004) were studied by RT-qPCR using TaqMan Low Density Array (TLDA). Twenty of the 40 tumors presented the RES phenotype (FIG. 9). Eighteen of the 20 tumors with the RES phenotype presented the CIS signature, whereas only two of the 20 tumors without the RES phenotype presented the CIS signature. Mutation of FGFR3, known to be associated with the second (Ta or non CIS) pathway, was found very rarely in tumors with the RES phenotype (only one case) and frequently in tumors without the RES phenotype (14 out of 20 tumors). As expected, the three normal samples did not present either the RES phenotype or the CIS signature. Tumors with the RES phenotype had a significantly higher expression of EZH2 (p=0.01) (data not shown).

Trichostatin a Strongly Inhibits the Growth of Bladder Cancer Cell Lines with the RES Phenotype

The findings described above have shown that the RES phenotype is associated with a subgroup of invasive tumors, and that the phenotype corresponds to the silencing of regions by H3K9 and K27 methylation and histone H3K9 hypoacetylation, but not DNA promoter methylation. TSA was used to treat a panel of bladder cancer-derived cell lines representative of the diversity of bladder tumors to determine whether the regional epigenetic silencing was restricted to a subset of bladder cancer cell lines (just as it was restricted to a subset of tumor samples). Two cell lines derived from well-differentiated tumors (MGHU3, which is mutated for FGFR3, and RT112) and four cell lines derived, like CL1207, from high-grade tumors (T24, TCCSUP, HT1376 and JMSU1, none mutated for FGFR3, and only T24 mutated for HRAS (Saison-Behmoaras et al., 1991)) were used. HRAS mutations, like FGFR3 mutations, are thought to be associated with the Ta progression pathway (FIG. 8c) (Jebar et al., 2005; Zhang et al., 2001). NHU cells were also included in the analysis.

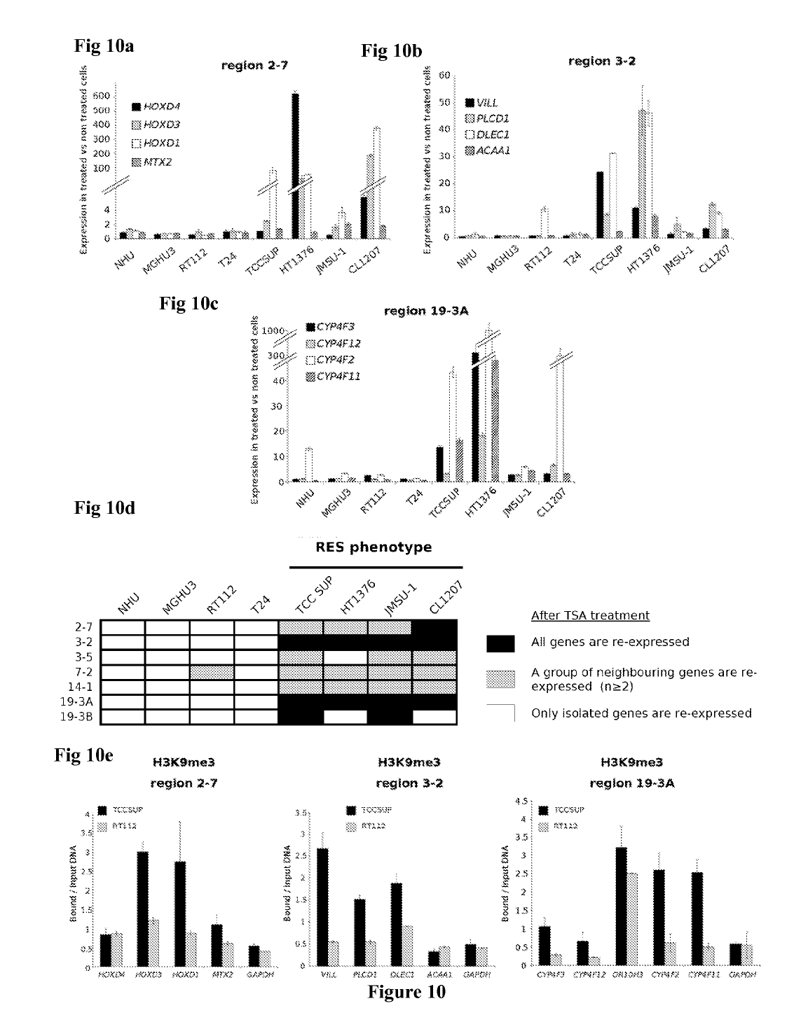

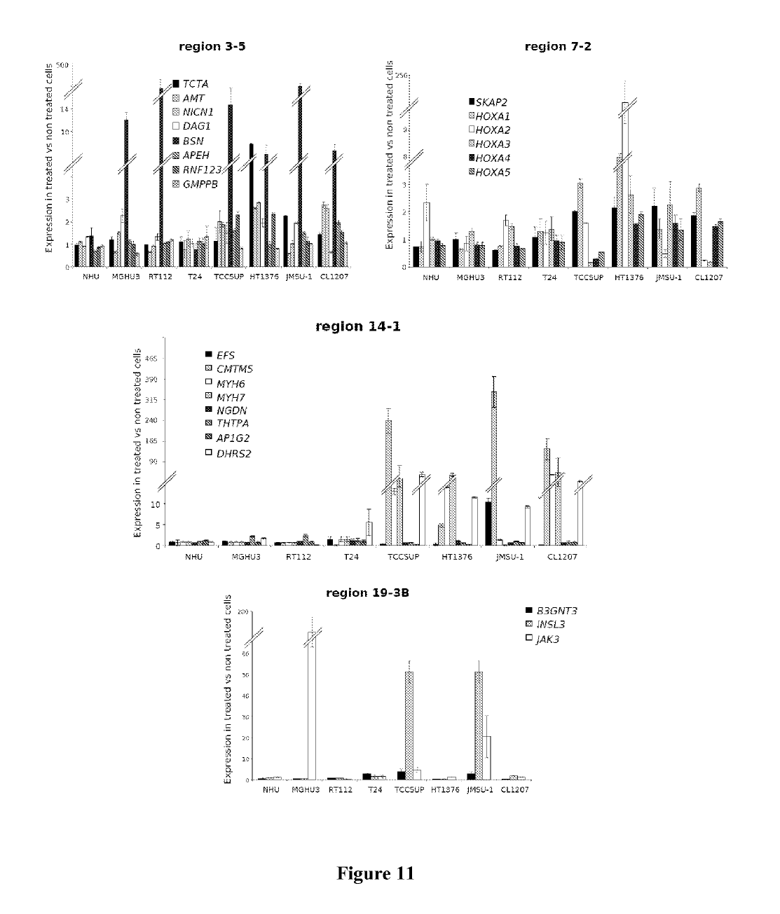

The effect of TSA was first investigated on re-expression of the genes within the seven epigenetic regions defining the RES phenotype. Re-expression results for three regions (2-7, 3-2 and 19-3A) are shown in FIG. 10a to c. The results for the other four regions are shown in FIG. 11. A summary of the effects of treatment on the different cell lines is provided in FIG. 10d. Two groups of cell lines were clearly distinguished. In the first group (NHU, MGHU3, RT112 and T24), most of the genes were not re-expressed, except for a few isolated genes in some cell lines. The second group of cell lines (TCCSUP, HT1376 and JMSU1) behaved like CL1207: gene re-expression was observed for most of the silenced regions after treatment. Definition of the re-expressed regions differed slightly between cell lines, as shown for region 2-7 in FIG. 10a: in CL1207, the epigenetic alteration affected HOXD4, HOXD3, HOXD1 and MTX2; in HT1376 it affected HOXD4, HOXD3 and HOXD1; in JMSU-1, it encompassed HOXD3, HOXD1 and MTX2; and in TCCSUP, it affected only HOXD3 and HOXD1.

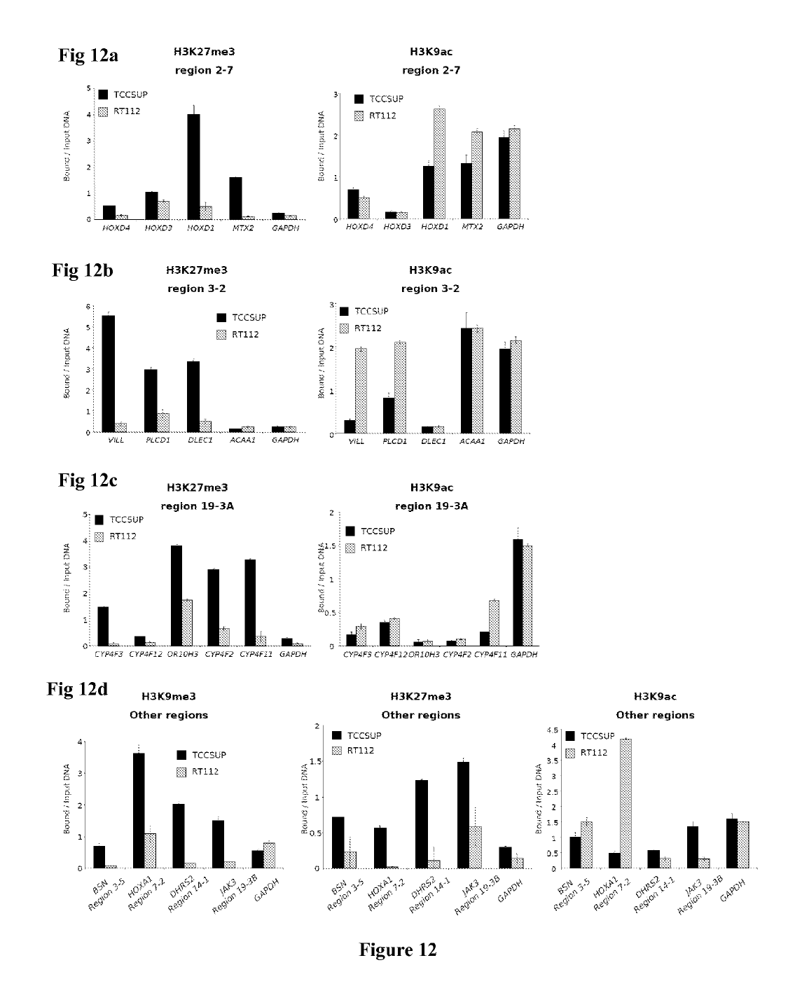

ChIP experiments were also carried out on three regions in detail (2-7, 3-2 and 19-3A) and for one gene in each of the other regions (3-5, 7-2, 14-1 and 19-3B) in the TCCSUP cell line, where all regions were re-expressed after TSA treatment and in RT112 cells, where no region was re-expressed, except two genes in region 7-2. For all seven regions, high levels of trimethylation of lysines 9 and 27 were observed in TCCSUP, but no significant trimethylation of either lysine 9 or 27 in RT112 (FIG. 10e and FIG. 12a to d). It should be noted that in region 19-3A, OR10H3, which was not expressed in normal or tumor samples, showed histone methylation in both TCCSUP and RT112 cell lines (FIG. 10e). Levels of acetylation on lysine 9 were higher in RT112 for some genes (FIG. 12a to d). Trimethylation of lysines 9 and 27 clearly differentiated cancer cells with the RES phenotype, such as TCCSUP and CL1207 cells, from normal (NHU) cells and other cancer cells (RT112 cells).

Thus, the bladder tumor cell lines, like tumor samples (FIG. 8), fell into two groups: one with frequent regional epigenetic silencing and the other without. The RES phenotype was associated with most of the high-grade tumor cells studied (JMSU1, HT1376 and TCCSUP, but not T24), but not with well-differentiated cancer cells (MGHU3 and RT112) or with normal (NHU) cells.

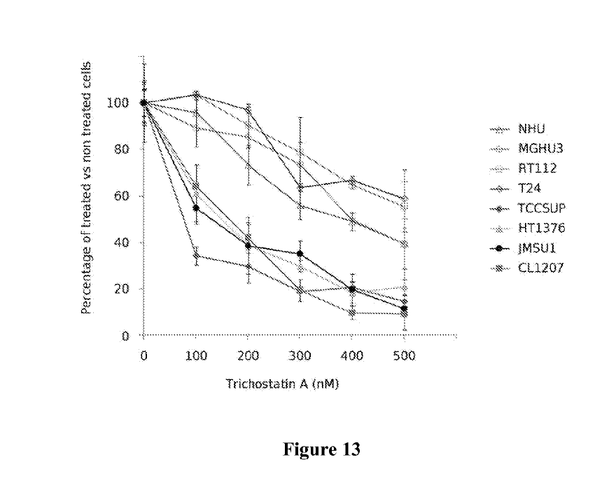

The RES phenotype was characterized by strong histone K9 and K27 methylation and K9 hypoacetylation, but extremely rare DNA methylation. Therefore, the growth inhibiting effects of TSA—a histone deacetylase inhibitor, which indirectly inhibits histone methylation—were compared on various cell lines with and without the RES phenotype (FIG. 13). Remarkably, the IC₅₀ (half maximal inhibitory concentration) of TSA was very different between the cell lines: 100 nM on average for cell lines with the RES phenotype (TCCSUP, HT1376, JMSU1 and CL1207) and 500 nM for the other cell lines (MGHU3, RT112 and T24) and NHU cells. This difference in sensitivity was not related to differences in doubling time between the two groups: NHU cells and all cancer cell lines except T24 (20 h) had doubling times of between 30 and 40 h.

### CONCLUSION

Using a combination of bioinformatics and experimental approaches, the inventors have defined seven chromosomal regions that can be simultaneously silenced in cancer. The silencing occurred in association with histone H3K9 hypoacetylation and H3K9 and K27 hypermethylation of promoter regions, mimicking the formation of facultative heterochromatin domains. Trichostatin A enabled gene re-expression and reversal of histone marks, clearly implicating the histone modifications in the silencing process. The demonstration that these regions were silenced simultaneously in the same set of tumors reveals, for the first time, the existence of a regional epigenetic silencing (RES) phenotype in cancer. The tumors with the RES phenotype are those tumors belonging to one of the two pathways of bladder tumor progression, the CIS pathway, which is responsible for the majority of invasive bladder tumors.

### Example 2

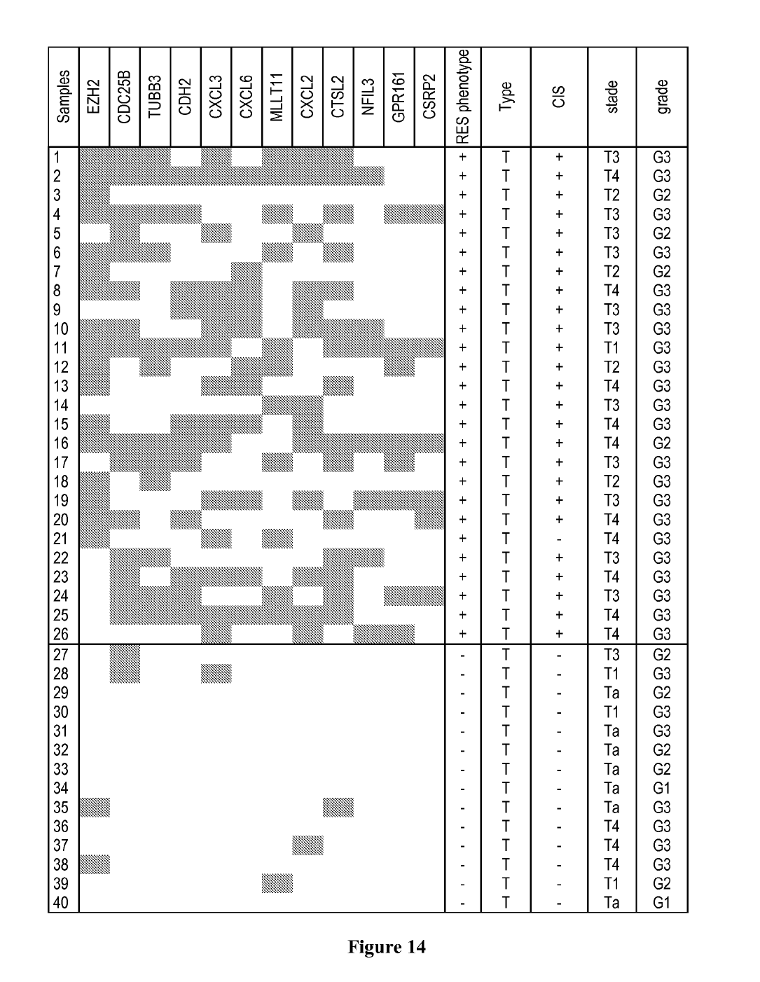

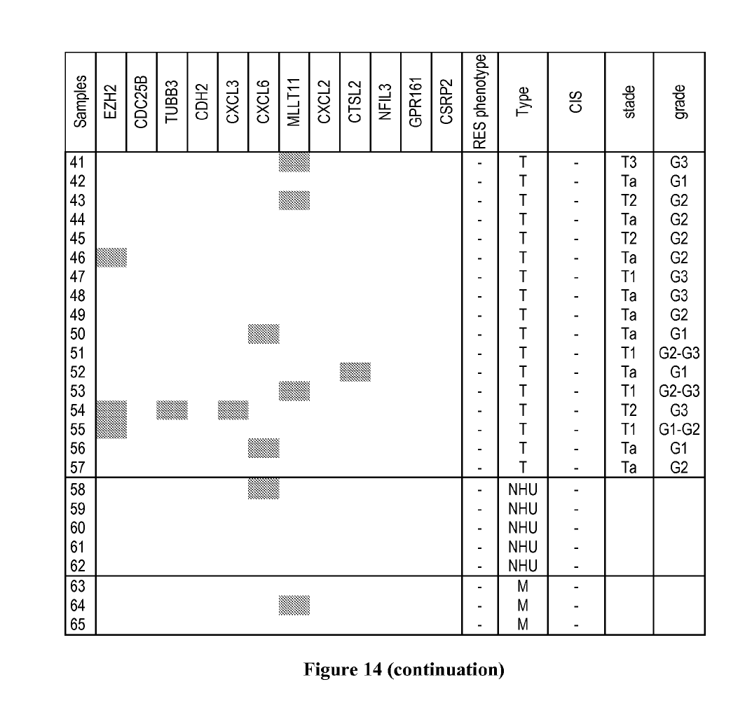

Affymetrix array expression was used to find markers for the RES phenotype. For all analyses, Affymetrix MASS signal values were Log 2-transformed and normalized by removing chip-specific and probe set-specific effects (the mean signal for all probe sets across one chip and the mean signal for one probe set across all chips, respectively). Statistical analysis and numerical calculations were carried out with Amadea® (Isoft, Gif-sur-Yvette, France). A SAM analysis (Tusher et al., PNAS 2001) was first performed between tumors with RES phenotype and invasive tumors without the RES phenotype. This analysis was restricted to the genes upregulated in the samples with RES phenotype with q-value<0.05. Genes with a fold-change above 2 was first selected. Then, the expression in the tumors with RES was compared with the normal urothelium samples and the muscle samples. Genes for which: 1) the signal was in average two times higher in the RES tumors compared to the normal samples, and 2) the signal was higher in the tumors with RES phenotype than in the muscle, were selected. 11 markers were obtained. EZH2 which was studied with RT-qPCR and found to be significantly more highly expressed in the tumors with RES phenotype was added. All markers and the expression of these markers in tumor samples compared to normal and muscle samples are presented in FIG. 14.

### Example 3

### Materials and Methods

### Patients and Tissue Samples

150 tumors were used to study gene expression. These carcinomas were obtained from patients included between 1988 and 2001 in the prospective database established in 1988 at the Department of Urology of Henri Mondor Hospital. Four normal urothelial samples, obtained as previously described were also used for transcriptome analysis. 40 of the 150 tumor samples and three normal samples were analyzed by RT-qPCR with TLDA format (Applied Biosystems, Courtaboeuf, France). All patients provided informed consent and the study was approved by the ethics committees of the different hospitals.

### RNA and DNA Extraction

RNA and DNA were extracted from the samples by cesium chloride density centrifugation. RNA and DNA were extracted from cell lines with Qiagen extraction kits (Qiagen, Courtaboeuf, France).

### Quantitative RT-PCR

1 μg of total RNA was used for reverse transcription, with random hexamers (20 pmol) and 200 U MMLV reverse transcriptase. To assess mRNA levels by real-time quantitative PCR (RT-qPCR), TaqMan Low Density Array (TLDA) was used on an ABI PRISM 7900 real-time thermal cycler (Applied Biosystems). All samples were run in duplicate and the reference 18S was used. Amounts of mRNAs of the genes of interest were normalized to that of the reference gene according to the 2^{-ΔCt} method.

Sorting Tumors with/without Regional Epigenetic Silencing (RES) Phenotype

To analyze which samples displayed the RES phenotype, the method described in example 1 was used.

### Results

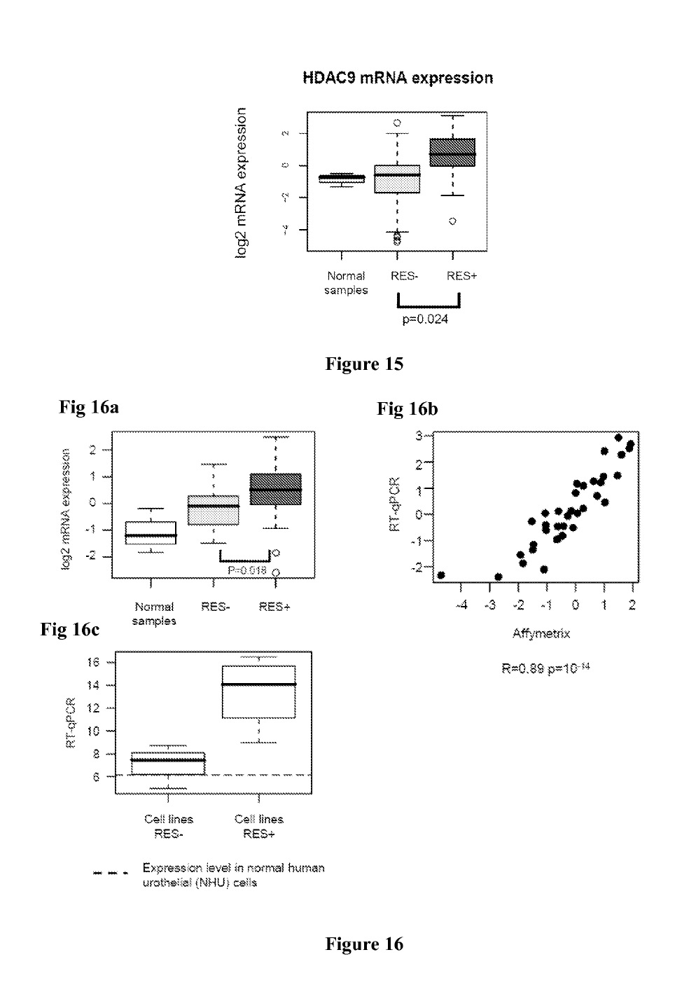

mRNA levels of histone deacetylases HDAC1, 2, 3, 4, 5, 6, 7, 8 and 9 were compared between invasive tumors with and without RES phenotype. The inventors found that HDAC9 was significantly (p<0.05) over-expressed in invasive tumors with RES phenotype compared to normal samples and to invasive tumors without RES phenotype (FIG. 15). The expression levels of others HDACs were identical in normal samples and tumors with or without RES phenotype (data not shown).

### Example 4

### Materials and Methods

### Patients and Tissue Samples

Patients and tissue samples were provided as described in example 3.

### RNA and DNA Extraction

RNA and DNA extraction were performed as described in example 3.

Cell Culture and siRNA Transfection

The bladder cancer cell line CL1207 was cultured in DMEM F-12 Glutamax medium supplemented with 10% FCS. Cells were transfected using Lipofectamine RNAiMAX (Invitrogen) with siRNA targeted against EZH2, and a scrumble siRNA as a negative control. Gene expression analyses and ChiP experiments were carried out 80 hours after transfection. Normal human urothelial (NHU) cells were established as finite cell lines and cultured in complete keratinocyte serum-free medium, as described (De Boer et al., 1997).

### Quantitative RT-PCR

1 μg of total RNA was used for reverse transcription, with random hexamers (20 pmol) and 200 U MMLV reverse transcriptase. To assess mRNA levels by real-time quantitative PCR (RT-qPCR), individual assays were used for the cell line experiments and the TaqMan Low Density Array (TLDA) was used for tumor samples, both on an ABI PRISM 7900 real-time thermal cycler (Applied Biosystems). With both methods, all samples were run in duplicate and the same reference 18S was used. Amounts of mRNAs of the genes of interest were normalized to that of the reference gene according to the 2^{-ΔCt} method.

### Chromatin Immunoprecipitation

Chromatin immunoprecipitation (ChIP) assays were carried out as previously reported (Stransky et al., 2006) in duplicate for CL1207 cells with or without siRNA transfection. Chromatin was prepared with an enzymatic kit (Active Motif, Rixensart, Belgium). An extract of the original chromatin was kept as an internal standard (Input DNA). The complexes were immunoprecipitated with 4 μg of antibodies against trimethyl histone H3 (Lys27) (Upstate Biotechnology, Santa Cruz, USA). The amount of immunoprecipitated target was determined by real-time PCR, in duplicate.

### Affymetrix Array and TLDA Analyses

For Affymetrix array expression analyses, Affymetrix MASS signal values were Log 2-transformed and normalized by removing chip-specific and probe set-specific effects (the mean signal for all probe sets across one chip and the mean signal for one probe set across all chips, respectively). TLDA arrays were normalized using the 18S signal and by removing the mean signal for one taqman probe across all samples and Log 2-transformed. Statistical analysis and numerical calculations were carried out with R 2.6 (R Foundation for Statistical Computing) and Amadea® (Isoft, Gif-sur-Yvette, France).

Sorting Tumors with/without Regional Epigenetic Silencing (RES) Phenotype

The sorting of tumors with and without RES phenotype was performed as described in example 1.

### Results

EZH2 mRNA expression levels was compared in a wide tumor set (n=150) between invasive tumors with (n=74) and without (n=29) RES phenotype and normal urothelium samples (n=4). Tumors with RES phenotype were identified as described above. This analysis was limited to invasive tumors in order that differences in expression levels between RES positive and negative tumors would be attributable to the phenotype itself and not the heterogeneity of tumor stages between each group.

As shown in FIG. 16a, EZH2 is significantly more highly expressed in invasive tumors with RES phenotype than in invasive tumors without RES phenotype and in normal samples. EZH2 Affymetrix expression data was validated (FIG. 16b) and shown to be highly correlated to RT-qPCR measurements performed on 40 tumors of the initial tumor set r=0.89 (p=10⁻¹⁴). When studying 7 bladder cancer cell lines (all derived from invasive bladder tumors), it was also found that EZH2 was more highly expressed in cancer cell lines with RES phenotype than those without, which displayed an expression level closer to the one of normal human urothelial (NHU) cells (FIG. 16c).

The role of EZH2 overexpression was studied in vitro in a cell line with RES phenotype, CL1207. CL1207 is a bladder cancer cell line derived with few passages from an invasive bladder tumor (De Boer et al., 1997). A knockdown of EZH2 was performed using siRNA. The effects of the siRNA transfection were analyzed on two chromosomal regions involved in the RES phenotype, regions 2-7 (comprising HOXD4, HOXD3 and HOXD1 genes) and 3-2 (comprising VILL, PLCD1, DLEC1 and ACAA1 genes).

EZH2 is known to catalyze the addition of a trimethyl group on H3K27. Accordingly, the level of trimethylation on H3K27 was studied by ChIP assay. Moreover, EZH2 gene expression was monitored by RT-qPCR.

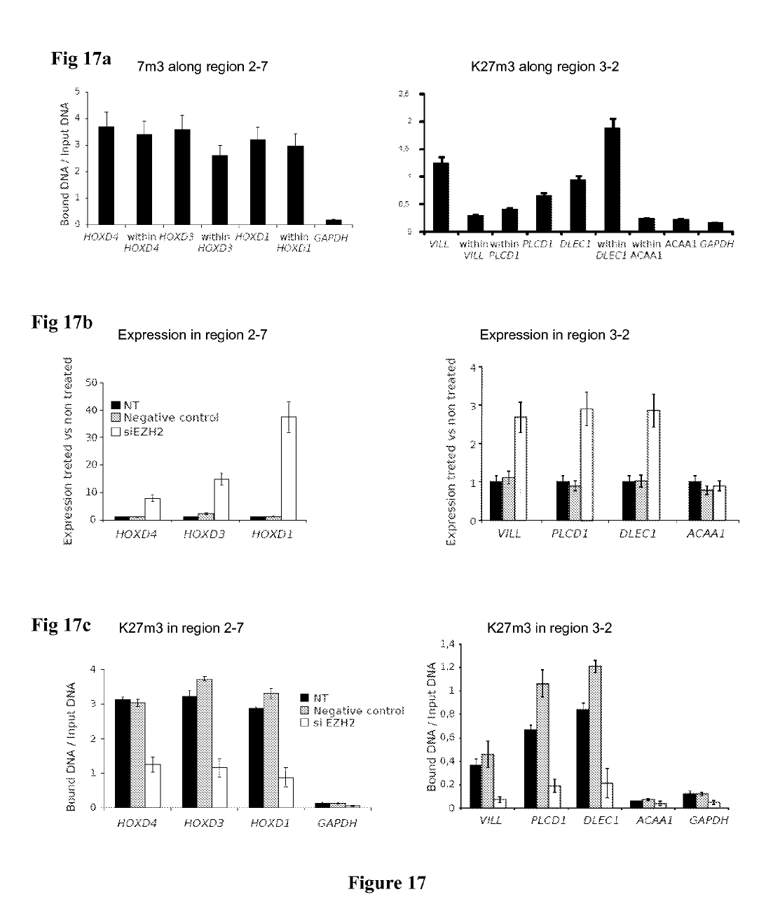

Initially, when genes of regions 2-7 and 3-2 were silenced, H3K27 was highly trimethylated along these regions in comparison to the promoter of a ubiquitously expressed gene GAPDH (FIG. 17a). The efficiency of the siRNA EZH2 knockdown was confirmed by RT-qPCR. Gene re-expression was induced specifically after EZH2 knockdown along the two regions 2-7 and 3-2 (FIG. 17b). By performing ChIP assay before and after transfection, it was observed that the re-expression of the genes after EZH2 knockdown corresponded to a decrease of H3K27me3 (FIG. 17c).

These results demonstrate that the inhibition of the histone methyltransferase EZH2 induces the re-expression of genes in silenced regions involved in the RES phenotype.

### Example 5

### MS275 and Gene Re-Expression in the Studied Regions

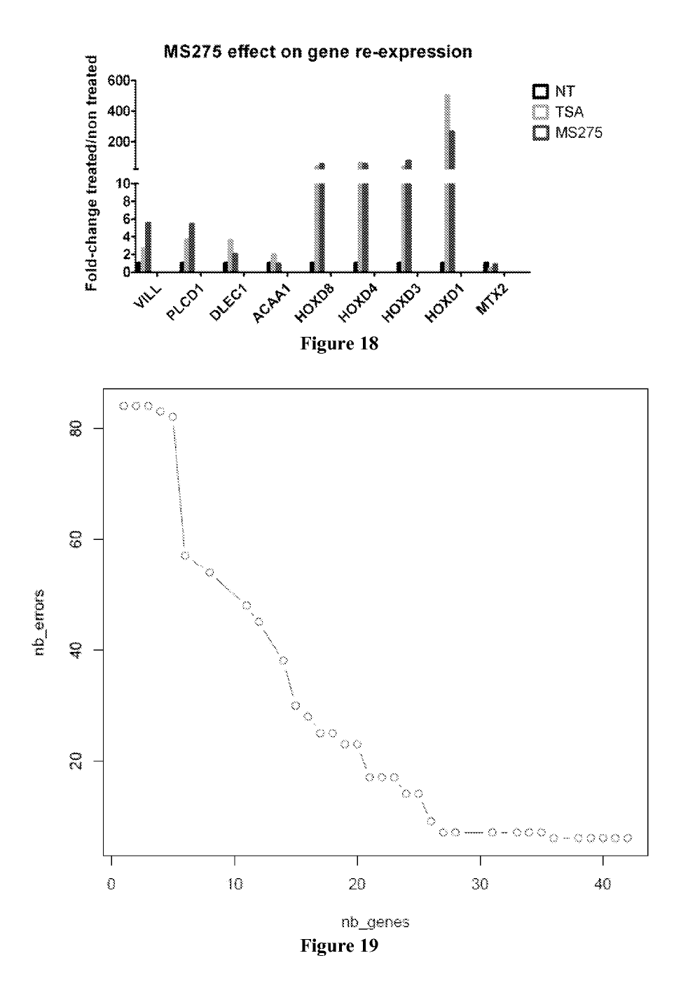

Trichostatin A targets all HDACs. To narrow down the list of HDACs potentially involved in the regulation of the repressed regions, the inventors used other inhibitors specific of one or several HDACs. They found that MS275, known for its inhibition of HDAC1, 2 and 3, enabled gene re-expression in the studied regions as well as did TSA (See FIG. 18). In FIG. 18, the study of mRNA expression in two repressed regions in the bladder cancer cell line CL1207 has been performed: regions on chromosome 3 (VILL to ACAA1) and 2 (HOXD8 to HOXD1). Therefore, it can be observed that inhibitors of HDAC1 and HDAC2, and less HDAC3 can be useful for reversing the gene repressions caused by the RES phenotype.

### Example 6

### Improved Markers for Determining the RES Phenotype of a Tumor

To improve the list of markers allowing the discrimination of the RES phenotype of a tumor, the inventors used a larger tumor set with better-quality chips. 157 bladder tumors were studied by Affymetrix Exon arrays. First, the inventors used a clustering approach to characterize the RES status of all tumors. They clustered tumors according to the expression level they displayed in all the regions characterizing the RES phenotype. Tumors were classified in two groups, RES+ (i.e., having the RES phenotype) or RES− (i.e., not having the RES phenotype). For further analyses, the inventors only kept the invasive tumors as most RES+ tumors are already invasive. The inventors wanted to identify positive markers of RES+ and RES− tumors, i.e. markers that are over-expressed in either group compared to the other group and to normal samples. To do so, they first selected all the genes of the array that answered these criteria (over-expressed by two-fold in one of the groups compared to the other group and to normal bladder samples). Then, the inventors performed a PAM analysis to study which set of these pre-selected genes could best classify the invasive tumors according to their RES status. A set of 50 markers (see below) enabled the classification of RES+/− tumors with a minimum error rate: when studying to the entire tumor set, the error rate was 2.5% (4 errors of classification for 157 tumors). This list can be limited to the first 27 markers (see FIG. 19), as the error rate is still minimal (3.2%).

| | | Characterized |
|---|---|---|
| Ranking | Gene | Group |
| 1 | ANXA10 | RES− |
| 2 | SLC16A1 | RES+ |
| 3 | SULF1 | RES+ |
| 4 | POSTN | RES+ |
| 5 | LOX | RES+ |
| 6 | FN1 | RES+ |
| 7 | CHI3L1 | RES+ |
| 8 | SFRP4 | RES+ |
| 9 | IGF2 | RES− |
| 10 | TNC | RES+ |
| 11 | COL3A1 | RES+ |
| 12 | FAP | RES+ |
| 13 | CXCL10 | RES+ |
| 14 | PLA2G7 | RES+ |
| 15 | GREM1 | RES+ |
| 16 | COL1A2 | RES+ |
| 17 | COL1A1 | RES+ |
| 18 | GUCY1A3 | RES+ |
| 19 | B3GALNT1 | RES− |
| 20 | PFTK1 | RES+ |
| 21 | COL6A3 | RES+ |
| 22 | FBN1 | RES+ |
| 23 | IFI30 | RES+ |
| 24 | CXCL9 | RES+ |
| 25 | PRRX1 | RES+ |
| 26 | AHNAK2 | RES+ |
| 27 | AEBP1 | RES+ |
| 28 | GBP5 | RES+ |
| 29 | MSN | RES+ |
| 30 | BGN | RES+ |
| 31 | CTHRC1 | RES+ |
| 32 | MMD | RES+ |
| 33 | C1S | RES+ |
| 34 | IGK@ | RES+ |
| 35 | COL5A2 | RES+ |
| 36 | THY1 | RES+ |
| 37 | C5orf13 | RES+ |
| 38 | EPHB6 | RES− |
| 39 | DSC2 | RES+ |
| 40 | SFRP2 | RES+ |
| 41 | NID2 | RES+ |
| 42 | TIMP2 | RES+ |
| 43 | SEMA6A | RES− |
| 44 | CXorf57 | RES− |
| 45 | SLC15A1 | RES− |
| 46 | HS6ST3 | RES− |
| 47 | KRT20 | RES− |
| 48 | ADAMTS12 | RES+ |
| 49 | GPX8 | RES+ |
| 50 | SULF2 | RES+ |

### REFERENCES

Barski A, Cuddapah S, Cui K, Roh T Y, Schones D E, Wang Z, Wei G, Chepelev I, Zhao K. High-resolution profiling of histone methylations in the human genome. Cell. 2007 May 18; 129(4):823-37
Billerey, C. et al. Frequent FGFR3 mutations in papillary non-invasive bladder (pTa) tumors. Am J Pathol, 2001, 158, 1955-9
Chirgwin, J. M., Przybyla, A. E., MacDonald, R. J. & Rutter, W. J. Isolation of biologically active ribonucleic acid from sources enriched in ribonuclease. Biochemistry, 1979, 18, 5294-9
De Boer, W I et al. Expression and functions of EGF, FGF and TGFbeta-growth-factor family members and their receptors in invasive human transitional-cell-carcinoma cells. Int J Cancer. 1997 Apr. 10; 71(2):284-91.
Diez de Medina, S. G. et al. Decreased expression of keratinocyte growth factor receptor in a subset of human transitional cell bladder carcinomas. Oncogene, 1997, 14, 323-30
Dyrskjot, L. et al. Gene expression in the urinary bladder: a common carcinoma in situ gene expression signature exists disregarding histopathological classification. Cancer Res, 2004, 64, 4040-8
Eisen, M. B., Spellman, P. T., Brown, P. O. & Botstein, D. Cluster analysis and display of genome-wide expression patterns. Proc Natl Acad Sci USA, 1998, 95, 14863-8
Frigola et al. Epigenetic remodelling in colorectal cancer results in coordinate gene suppression across an entire chromosome band. Nat. Genet. 2002, 38, 540-549
Greiner D, Bonaldi T, Eskeland R, Roemer E, Imhof A. Identification of a specific inhibitor of the histone methyltransferase SU(VAR)3-9. Nat Chem. Biol. 2005 August; 1(3):143-5.
Jebar, A. H. et al. FGFR3 and Ras gene mutations are mutually exclusive genetic events in urothelial cell carcinoma. Oncogene, 2005, 24, 5218-25
Koch C M, Andrews R M, Flicek P, Dillon S C, Karaoz U, Clelland G K, Wilcox S, Beare D M, Fowler J C, Couttet P, James K D, Lefebvre G C, Bruce A W, Dovey O M, Ellis P D, Dhami P, Langford C F, Weng Z, Birney E, Carter N P, Vetrie D, Dunham I. The landscape of histone modifications across 1% of the human genome in five human cell lines. Genome Res. 2007 June; 17(6):691-707
Kubicek S, O'Sullivan R J, August E M, Hickey E R, Zhang Q, Teodoro M L, Rea S, Mechtler K, Kowalski J A, Homon C A, Kelly T A, Jenuwein T. Reversal of H3K9me2 by a small-molecule inhibitor for the G9a histone methyltransferase. Mol. Cell. 2007 Feb. 9; 25(3):473-81
Novak, P. et al. Epigenetic inactivation of the HOXA gene cluster in breast cancer. Cancer. Res. 2006, 66, 10664-70.
Reyal, F. et al. Visualizing chromosomes as transcriptome correlation maps: evidence of chromosomal domains containing co-expressed genes—a study of 130 invasive ductal breast carcinomas. Cancer Res, 2005, 65, 1376-83
Saison-Behmoaras, T. et al. Short modified antisense oligonucleotides directed against Ha-ras point mutation induce selective cleavage of the mRNA and inhibit T24 cells proliferation. Embo J, 1991, 10, 1111-8
Siedlecki P. et al., Discovery of two novel, small-molecule inhibitors of DNA methylation. J Med. Chem. 2006 Jan. 26;49(2):678-83
Southgate, J., Hutton, K. A., Thomas, D. F. & Trejdosiewicz, L. K. Normal human urothelial cells in vitro: proliferation and induction of stratification. Lab Invest, 1994, 71, 583-94
Stransky, N. et al. Regional copy number-independent deregulation of transcription in cancer. Nat. Genet. 2006, 38, 1386-96
Tusher V G, Tibshirani R, Chu G. Significance analysis of microarrays applied to the ionizing radiation response. Proc Natl Acad Sci USA. 2001 Apr. 24; 98(9):5116-21.
Van Oers, J. M. et al. A simple and fast method for the simultaneous detection of nine fibroblast growth factor receptor 3 mutations in bladder cancer and voided urine. Clin Cancer Res, 2005, 11, 7743-8
Whetstine J. et al., Reversal of Histone Lysine Trimethylation by the JMJD2 Family of Histone Demethylases. Cell, Volume 125, Issue 3, Pages 467-481
Wu, X. R. Urothelial tumorigenesis: a tale of divergent pathways. Nat Rev Cancer, 2005, 5, 713-25
Xiong, Z. & Laird, P. W. COBRA: a sensitive and quantitative DNA methylation assay. Nucleic Acids Res, 1997, 25, 2532-4
Zhang, Z. T. et al. Role of Ha-ras activation in superficial papillary pathway of urothelial tumor formation. Oncogene, 2001, 20, 1973-80

1-20. (canceled)

21. An in vitro method for determining the regional epigenetic silencing (RES) phenotype of a tumor, wherein the method comprises determining the expression level of at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5orf13, DSC2, SFRP2. NID2, TIMP2, ADAMTS12, GPX8 and SULF2, and wherein the over-expression of said genes is indicative of the RES phenotype of the tumor.

22. The method according to claim 21, wherein the method further comprises determining the expression level of at least 3 genes selected from the group consisting of ANXA10, IGF2, B3GALNT1, EPHB36, SEMA6A, CXorf57, SLC15A1, HS6ST3 and KRT20, and wherein the absence of over-expression of said genes is indicative of the RES phenotype of the tumor or confirms its RES phenotype.

23. The method according to claim 21, wherein said tumor is a bladder tumor.

24. An in vitro method for determining the RES phenotype of a tumor, wherein the method comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed and/or determining the number of chromosomal regions selected from the group consisting of regions 2-7, 3-2, 3-5, 7-2, 14-1, 19-3A and 19-3B which are silenced, and optionally assessing the expression level of the EZH2 histone methyltransferase in said tumor, and wherein the RES phenotype is defined either by the presence of at least three of said over-expressed genes and/or by the presence of at least three of said silenced regions, and/or by the presence of at least two of said silenced regions and an overexpression of the EZH2 histone methyltransferase.

25. The method according to claim 24, wherein the method comprises determining the number of chromosomal regions selected from the group consisting of regions 2-7, 3-2, 3-5, 7-2, 14-1, 19-3A and 19-3B which are silenced in said tumor, and wherein the RES phenotype is defined by the presence of at least three of said silenced regions.

26. The method according to claim 24, wherein the method comprises determining the number of chromosomal regions selected from the group consisting of regions 2-7, 3-2, 3-5, 7-2, 14-1, 19-3A and 19-3B which are silenced, and assessing the expression level of the EZH2 histone methyltransferase in said tumor, and wherein the RES phenotype is defined by the presence of at least two of said silenced regions and an overexpression of the EZH2 histone methyltransferase.

27. The method according to claim 24, wherein the method comprises determining the number of genes selected from the group consisting of EZH2, CDC25B, TUBB3, CDH2, CXCL3, CXCL6, MLLT11, CXCL2, CTSL2, NFIL3, GPR161, CSRP2 and HDAC9 which are over-expressed, and wherein the RES phenotype is defined by the presence of at least three of said over-expressed genes.

28. The method according to claim 24, wherein said tumor is a bladder tumor.

29. An in vitro method for diagnosing an aggressive tumor in a subject, wherein the method comprises determining the RES phenotype in a tumor according to the method of claim 21, and wherein the presence of the RES phenotype in said tumor is indicative of an aggressive tumor.

30. The method according to claim 29, wherein said tumor is a bladder tumor belonging to the CIS pathway.

31. The method according to claim 29, wherein said tumor is a bladder tumor which is a muscle-invasive or high grade tumor.

32. An in vitro method for predicting the sensitivity of a tumor to an epigenetic therapy, wherein the method comprises determining the RES phenotype in said tumor according to the method of claim 21, and wherein the presence of the RES phenotype in said tumor is predictive that said tumor is sensitive to an epigenetic therapy.

33. The method according to claim 32, wherein the epigenetic therapy comprises at least one compound selected from the group consisting of histone deacetylase inhibitors, histone methyltransferase inhibitors and histone demethylases, and any combination thereof, optionally in combination with a DNA methyltransferase inhibitor.

34. The method according to claim 32, wherein said tumor is a bladder tumor.

35. An in vitro method for predicting the sensitivity of a tumor to an epigenetic therapy, wherein the method comprises determining the RES phenotype in said tumor according to the method of claim 24, and wherein the presence of the RES phenotype in said tumor is predictive that said tumor is sensitive to an epigenetic therapy.

36. An in vitro method for selecting a patient affected with a tumor for an epigenetic therapy or determining whether a patient affected with a tumor is susceptible to benefit from an epigenetic therapy, wherein the method comprises determining the RES phenotype of said tumor according to the method of claim 21, and wherein the presence of the RES phenotype in said tumor is predictive that an epigenetic therapy is indicated for said patient.

37. The method according to claim 36, wherein said tumor is a bladder tumor.

38. The method according to claim 36, wherein the epigenetic therapy comprises at least one compound selected from the group consisting of histone deacetylase inhibitors, histone methyltransferase inhibitors and histone demethylases, and any combination thereof, optionally in combination with a DNA methyltransferase inhibitor.

39. An in vitro method for selecting a patient affected with a tumor for an epigenetic therapy or determining whether a patient affected with a tumor is susceptible to benefit from an epigenetic therapy, wherein the method comprises determining the RES phenotype of said tumor according to the method of claim 24, and wherein the presence of the RES phenotype in said tumor is predictive that an epigenetic therapy is indicated for said patient.

40. A method for treating a cancer in a patient affected with a tumor with a RES phenotype comprising administrating an epigenetic compound to said patient.

41. The method according to the claim 40, wherein said epigenetic compound is selected from the group consisting of histone deacetylase inhibitor, histone methyltransferase inhibitor and histone demethylase, and any combination thereof, optionally in combination with a DNA methyltransferase inhibitor or another antineoplastic agent.

42. The method according to claim 41, wherein said epigenetic compound is an inhibitor of histone deacetylases HDAC1, HDAC2 and/or HDAC3.

43. The method according to claim 40, wherein said tumor is selected from the group consisting of bladder cancer, colorectal cancer, oesophageal cancer, neuroblastoma, breast cancer and lung cancer, preferably from the group consisting of bladder cancer, colorectal cancer and breast cancer.

44. The method according to claim 43, wherein said tumor is a bladder tumor.

45. A kit for determining the RES phenotype of a tumor, wherein the kit comprises detection means selected from the group consisting of a pair of primers, a probe and an antibody specific to a) at least 20 genes selected from the group consisting of SLC16A1, SULF1, POSTN, LOX, FN1, CHI3L1, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY, C5orf13, DSC2, SFRP2, NID2, TIMP2, ADAMTS12, GPX8, and SULF2; or b) the genes EZH12, CDC25B, TUBB3, CDH2, CXCL3, CXCL6, MLLT11, CXCL2, CTSL2, NFIL3, GPR161, CSRP2 and HDAC9.

46. A DNA chip for determining the RES phenotype of a tumor, wherein the DNA chip comprises a solid support which carries nucleic acids that are specific to a) at least 20 genes selected from the group consisting of SLC16A1, SULF1, POSTN, LOX, FN1, CHI3L, SFRP4, TNC, COL3A1, FAP, CXCL10, PLA2G7, GREM1, COL1A2, COL1A1, GUCY1A3, PFTK1, COL6A3, FBN1, IFI30, CXCL9, PRRX1, AHNAK2, AEBP1, GBP5, MSN, BGN, CTHRC1, MMD, C1S, IGK@, COL5A2, THY1, C5 orf13, DSC2, SFRP2, NID2, TIMP2, ADAMTS12, GPX8, and SULF2; or b) the genes EZH2, CDC25B, TUBB3, CDH2, CXCL3, CXCL6, MLLT11, CXCL2, CTSL2, NFIL3, GPR161, CSRP2 and HDAC9.
